(19) United States
(12) Patent Application Publication
(10) Pub. No.: US 2009/0018146 A1
(43) Pub. Date: Jan. 15, 2009

(54) Combination Therapy with Triterpenoid Compounds and Proteasome Inhibitors

(75) Inventors: Jordan Gutterman, Houston, TX (US); Amos Gaikwad, Sugarland, TX (US); Ann Poblenz, Playa Del Rey, CA (US); Valsala Haridas, Pearland, TX (US)

Correspondence Address: FULBRIGHT & JAWORSKI L.L.P., 600 CONGRESS AVE., SUITE 2400, AUSTIN, TX, 78701, US

(73) Assignee: Research Development Corporation, Carson City, NV (US)

(21) Appl. No.: 11/814,268

(22) PCT Filed: Jan. 26, 2006
(86) PCT No.: PCT/US2006/002821

§ 371 Date: Aug. 8, 2008

Related U.S. Application Data
(60) Provisional application No. 60/647,513, filed on Jan. 27, 2005.

(51) Int. Cl. A61K 31/4965; A61P 35/00

(52) U.S. Cl. 514/255.06

(57) ABSTRACT

The present invention provides therapeutic compositions comprising a natural triterpenoid and a proteasome inhibitor. These compositions will be particularly useful in the treatment of malignancies and inflammation. The present invention also provides methods of treating a subject having a malignancy or an inflammatory disorder comprising administering to the subject a natural triterpenoid and a proteasome inhibitor.

### BACKGROUND OF THE INVENTION

1. Field of the Invention

The present invention relates generally to the field of medicine. More specifically, the invention relates to the treatment of malignancies and inflammation using combinations of triterpenes and proteasome inhibitors.

2. Description of the Related Art

Stress is a fundamental aspect of cellular life. Thus, the ability to cope with various environmental or internal stressors is essential for the maintenance and survival of organisms (McClintock, 1984). One of the early characteristics of resistance or tolerance to stress is activation of the heat shock proteins (Hsps), which can be traced in evolution to the earliest prokaryotes, including archea (Feder and Hofmann, 1999). Since Hsps promote cell survival in multi-cellular organisms, elimination of damaged or mutated cells may become compromised when Hsps are continuously activated.

During neoplastic transformation, cells activate a stress response to protect themselves against elimination (Benhar et al., 2002). As a consequence, cancer cells are eventually selected for their anti-apoptotic phenotype. Activation of Hsps in various cancers is common and is responsible, in part, for the anti-apoptotic phenotype of cancer cells and contributes to resistance to anticancer drugs (Creagh et al, 2000; Jolly and Morimoto, 2000; Beere and Green, 2001).

Of the known mechanisms of acquired resistance to apoptosis, over-expression of the major stress-inducible family of heat shock proteins (Hsps) (Creagh et al, 2000) is prominent. Hsp70 interacts with apoptotic protease activating factor-1 (Apaf-1) (Saleh et al, 2000; beere et al, 2000), the apoptosis inducing factor (AIF) (Ravagnan et al., 2001), and negatively interferes with the caspase dependent and independent process of apoptosis (Creagh et al., 2000). Besides Hsps, a class of proteins called the inhibitor of apoptosis (IAP) proteins block cell death by inhibiting upstream and terminal caspases (Yang and Wu, 2003). Amongst the eight known mammalian IAPs, the XIAP appears to be most potent (Ki in the low nM range) and best characterized, with its ability to inhibit activated caspases 3, 7 and 9 (reviewed in references Yang and Yu, 2003; Holcik et al., 2001).

In general, elevated levels of Hsps (Creagh et al., 2000) and XIAP (Yang and Yu, 2003; Holcik et al., 2001) are associated with drug resistance and poor prognosis. Down-regulation of Hsps (Nylandsted et al., 2000; Nylandsted et al., 2002) and XIAP (Tamm et al., 2000) by anti-sense and other interventions such as 17-AAG (an inhibitor of Hsp90) (13) demonstrate the ability to overcome apoptotic resistance.

Specific inhibitors of the proteasome have been shown to induce apoptosis and reduce inflammation. In some cases, however, resistance to the proteasome inhibitor eventually develops. The inhibition of proteasomal function is a potent stimulus of the heat shock protein response, likely due to the accumulation of undegraded proteins. As mentioned above, acquired resistance to apoptosis is a hallmark of most types of cancer, and overexpression of heat shock proteins is a prominent mechanism of acquired resistance to apoptosis. Therefore, there is a need for improved methods and compositions for the treatment of cancer and inflammation.

### SUMMARY OF THE INVENTION

In one embodiment, the present invention provides a method of inducing apoptosis in a malignant cell comprising contacting the malignant cell with a natural triterpenoid and a proteasome inhibitor. In another embodiment, the invention provides a method treating a subject with a malignancy comprising administering to the subject a natural triterpenoid and a proteasome inhibitor. In yet another embodiment, the invention provides a method treating a subject having inflammation comprising administering to the subject a natural triterpenoid and a proteasome inhibitor. The subject may be a mammal. In certain embodiments, the mammal is a human.

The present invention also provides a pharmaceutical composition comprising a natural triterpenoid and a proteasome inhibitor in a pharmacologically acceptable buffer, solvent or diluent. In one embodiment, the invention provides a method of treating a subject with a malignancy comprising administering to the subject a therapeutically effective amount of a pharmaceutical composition comprising a natural triterpenoid and a proteasome inhibitor in a pharmacologically acceptable buffer, solvent or diluent. In another embodiment, the invention provides a method of treating a subject having inflammation comprising administering to the subject a therapeutically effective amount of a pharmaceutical composition comprising a natural triterpenoid and a proteasome inhibitor in a pharmacologically acceptable buffer, solvent or diluent.

As used herein, a “natural triterpenoid” is a triterpenoid that is naturally produced in a living organism. This definition encompasses natural triterpenoids whether obtained from the natural source or synthesized. Non-limiting examples of, natural triterpenoids include asiatic acid; ursolic acid; celatrol; hederacolchiside-A1; lupeol; dehydroebriconic acid; oleanic acid; frondiside A; betulinic acid; friedelin; canophyllol; zeylanol; aradecoside I; and glycyrrhizinic acid.

In certain aspects of the invention, the natural triterpenoid is a plant-derived triterpenoid. A plant-derived triterpenoid is a natural triterpenoid that is derivable from a plant. As used herein, “derivable” means capable of being obtained or isolated. In some embodiments, the plant-derived triterpenoid is derivable from a plant of the genus Acacia. In one embodiment the triterpenoid is derivable from Acacia victoriae. The triterpenoid may be, for example, an avicin. Avicins are triterpenoid electrophilic metabolite molecules isolatable from the plant Acacia victoriae. Although any avicin is suitable, in specific embodiments the avicin is Avicin D, Avicin G, Avicin B, or a mixture thereof (see U.S. patent application Ser. No. 09/992,556, incorporated herein by reference).

The avicin may be further defined as a composition comprising a triterpene moiety attached to a monoterpene moiety having the molecular formula:

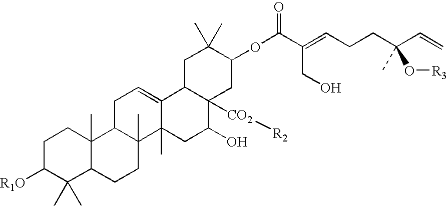

or a pharmaceutical formulation thereof, wherein a) R1 and R2 are selected from the group consisting of hydrogen, C1-C5 alkyl, and an oligosaccharide; b) R3 is selected from the group consisting of hydrogen, hydroxyl, C1-C5 alkyl, C1-C5 alkylene, C1-C5 alkyl carbonyl, a sugar, and a monoterpene group; and c) the formula further comprises R4, wherein R4 is selected from the group consisting of hydrogen, hydroxyl, C1-C5 alkyl, C1-C5 alkylene, C1-C5 alkyl carbonyl, a sugar, C1-C5 alkyl ester, and a monoterpene group, and wherein R4 may be attached to the triterpene moiety or the monoterpene moiety. In particular, R3 may be a sugar, such as one selected from the group consisting of glucose, fucose, rhamnose, arabinose, xylose, quinovose, maltose, glucuronic acid, ribose, N-acetyl glucosamine, and galactose. In specific embodiments, the avicin further comprises a monoterpene moiety attached to the sugar.

In additional embodiments, the compositions of the present invention comprise an avicin wherein R3 has the following formula:

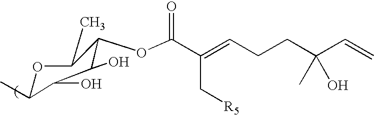

wherein R5 is selected from the group consisting of hydrogen, hydroxyl, C1-C5 alkyl, C1-C5 alkylene, C1-C5 alkyl carbonyl, a sugar, C1-C5 alkyl ester, and a monoterpene group. In particular embodiments, the R5 is a hydrogen or a hydroxyl. In other particular embodiments, the R1 and R2 each comprise an oligosaccharide, although in other embodiments each may comprise a monosaccharide, a disaccharide, a trisaccharide or a tetrasaccharide. In further specific embodiments, R1 and R2 each comprise an oligosaccharide comprising sugars that are separately and independently selected from the group consisting of glucose, fucose, rhamnose, arabinose, xylose, quinovose, maltose, glucuronic acid, ribose, N-acetyl glucosamine, and galactose. In specific embodiments, at least one sugar is methylated. The R4 may be attached to the triterpene moiety through one of the methylene carbons attached to the triterpene moiety, and in specific embodiments the triterpene moiety is oleanolic acid instead of acacic acid.

In particular embodiments of the invention, the compositions include an avicin further defined as comprising a triterpene glycoside having the molecular formula:

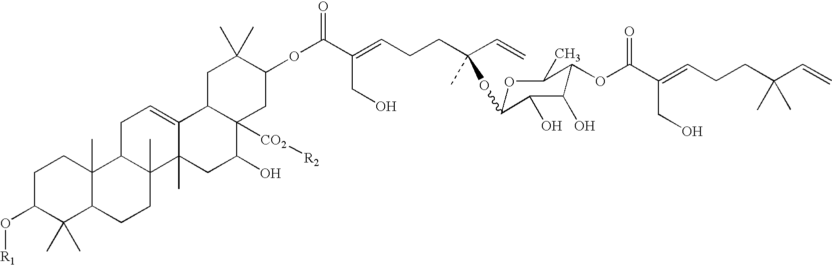

or a pharmaceutical formulation thereof, wherein a) R1 is an oligosaccharide comprising N-acetyl glucosamine, fucose and xylose; and b) R2 is an oligosaccharide comprising glucose, arabinose and rhamnose.

In other embodiments, the composition comprises an avicin having the molecular formula (Avicin D):

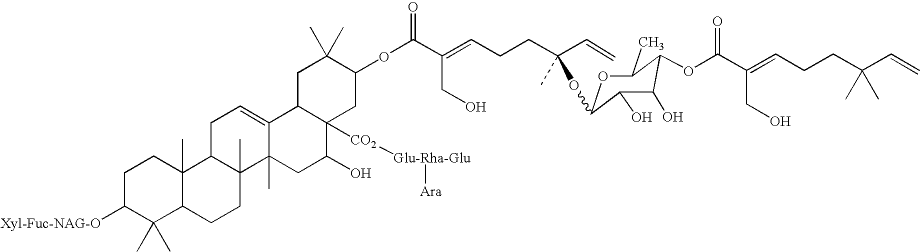

or a pharmaceutical formulation thereof.

In particular, the avicin is further defined as a triterpene glycoside having the molecular formula (Avicin G):

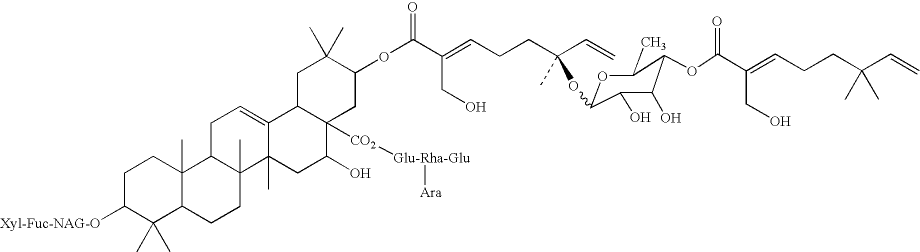

or a pharmaceutical formulation thereof wherein, a) R1 is an oligosaccharides comprising N-acetyl glucosamine, fucose and xylose; and b) R2 is an oligosaccharides comprising glucose, arabinose and rhamnose.

The avicin may have the molecular formula:

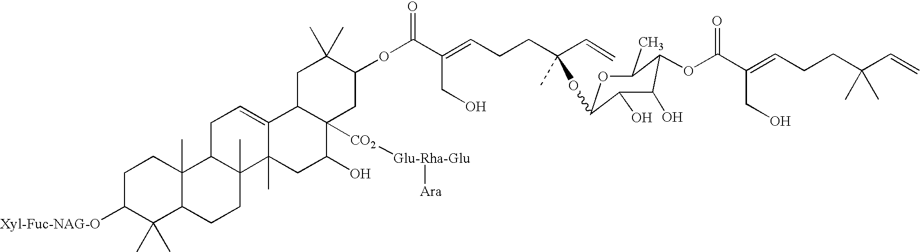

or a pharmaceutical formulation thereof. The avicin may be further defined as comprising a triterpene glycoside having the molecular formula:

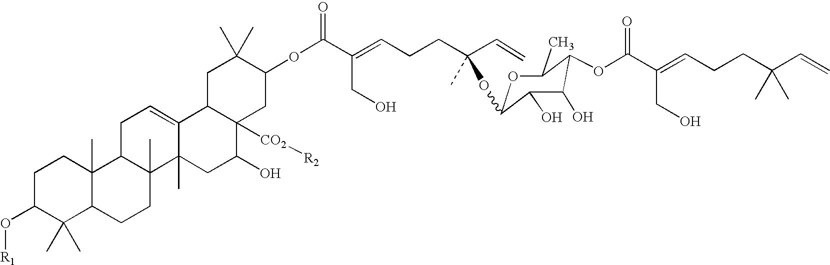

or a pharmaceutical formulation thereof, wherein, a) R1 is an oligosaccharide comprising N-acetyl glucosamine, glucose, fucose and xylose; and b) R2 is an oligosaccharide comprising glucose, arabinose and rhamnose. The avicin may be further defined as having the molecular formula (Avacin B):

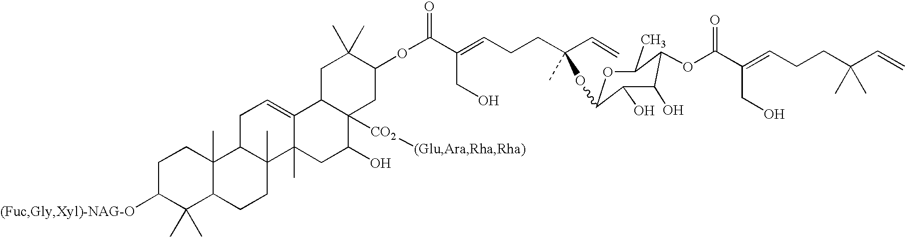

The avicin may be further defined as comprising a triterpene moiety, an oligosaccharide and three monoterpene units, and the triterpene moiety is acacic acid or oleanolic acid.

The proteasome inhibitor may be, for example, a peptide aldehyde, a peptide boronate, a peptide vinyl sulfone, a peptide epoxyketone, a lactacystin, or a lactacystin derivative. Specific examples of proteasome inhibitors include MG132, boronate MG132, MG262, boronate MG262, MG115, ALLN, PSI, CEP1612, epoxomicin, eponemycin, epoxyketone eponemycin, dihydroeponemycin, LLM, PS-341 (also known as bortezomib or Velcade®), DFLB, PS-273, ZLVS, NLVS, TMC-95A, lactacystin, β-lactone, gliotoxin, and EGCG. Additional examples of proteasome inhibitors are disclosed in Kisselev and Goldberg (2001) and Myung et al. (2001), both of which are incorporated herein in their entirety.

In certain embodiments, the malignant cell is an ovarian cancer cell, a pancreatic cancer cell, a renal cancer cell, a prostate cancer cell, a melanoma cell, or a leukemia cell. In certain preferred embodiments, the malignant cell may be of myeloid origin, such as a myeloma cells.

Thus, it will be understood, that in certain embodiments the invention concerns methods for treating a cell proliferative disease comprising administering an effective amount of a natural triterpenoid compound and an effective amount of a proteasome inhibitor. The term cell proliferative disease as used herein, comprises cancerous and precancerous conditions. For example, in certain cases methods according to the invention may be used to treat ovarian cancer, pancreatic cancer, renal cancer, prostate cancer, a melanoma, a leukemia, multiple myeloma or metastases thereof. It is further contemplated that the natural triterpenoid and the proteasome inhibitor may be administered simultaneously (either together or separately) or sequentially. Thus, in certain very specific embodiments methods according to the invention comprise a method for treating multiple myeloma comprising administering an effective amount of a natural triterpenoid molecule, such as ah avicin, and PS-341 (bortezomib).

In some embodiments, the subject has an inflammatory disorder. In certain aspects of the invention, the inflammatory disorder is an autoimmune disorder. Examples of autoimmune disorders that may be treated according to the present invention include rheumatoid arthritis, juvenile rheumatoid arthritis, osteoarthritis, psoriatic arthritis, atopic dermatitis, eczematous dermatitis, psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, leprosy reversal reactions, erythema nodosum leprosum, autoimmune uveitis, polychondritis, Stevens-Johnson syndrome, lichen planus, sarcoidosis, primary biliary cirrhosis, uveitis posterior, or interstitial lung fibrosis.

Administering the natural triterpenoid and the proteasome inhibitor may comprise any effective method including direct intratumoral injection, intravenous delivery, topical administration, or oral administration. Where the pharmaceutical composition is administered orally, the composition can be swallowed or inhaled. The malignancy or inflammation can be of any type that is treatable with the compounds of the invention. In particular embodiments of the invention the malignancy being treated is selected from the group consisting of ovarian cancer, pancreatic cancer, melanoma, prostate cancer, breast cancer, and leukemia.

The pharmaceutical composition may further comprise a targeting agent. The targeting agent may direct the triterpenoid and the proteasome inhibitor to a tumor cell and be chemically linked to said triterpenoid and said proteasome inhibitor. A suitable targeting agent comprises an antibody or an aptamer, which binds to the tumor cell.

In particular embodiments of the invention, the step of administering a therapeutically effective amount of a pharmaceutical composition comprising the triterpenoid and the proteasome inhibitor to treat cancer or inflammation comprises administering to a patient from about 1 mg/kg/day to about 100 mg/kg/day, about 3 mg/kg/day to about 75 mg/kg/day, about 5 mg/kg/day to about 50 mg/kg/day, or about 10 mg/kg/day to about 25 mg/kg/day of the pharmaceutical composition.

The pharmaceutical composition used to treat a subject with cancer may further comprise an additional agent capable of killing tumor cells, or any additional number of chemical agents. The method of treating cancer may additionally include the step of administering to the cancer patient at least a second pharmaceutical composition comprising at least a second composition capable of killing tumor cells. Additionally, the method may further comprise treating the cancer by tumor irradiation, and the radiation may be selected from the group consisting of X-ray radiation, UV-radiation, γ-radiation, or microwave radiation.

In still yet another aspect, the invention provides a method of treating a subject for a condition selected from the group consisting of high cholesterol, ulcers, fungal or viral infection, congestion, arrhythmia, hypertension or capillary fragility. In particular embodiments of the invention, the subject may be a human. In further embodiments of the invention, the step of administering comprises giving to a patient from about 1 mg/kg/day to about 100 mg/kg/day, about 3 mg/kg/day to about 75 mg/kg/day, about 5 mg/kg/day to about 50 mg/kg/day, or about 10 mg/kg/day to about 25 mg/kg/day of a pharmaceutical composition of the invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIGS. 1A and 1B: Regulation of Stress Proteins by Avicin D. Jurkat cells were treated with avicin D from 30 minutes up to 4 hours as described in Example 1. FIG. 1A shows the western blot analysis of cellular proteins (25 μg) from untreated (Un) and avicin D treated cells probed with various antibodies (Hsp70, Hsp90, Hsc70, Hsp60, Hsp27, Grp75, calnexin and β-actin). FIG. 1B shows densitometric values obtained from scanning the autoradiographic signals of the western blots and plotted as the percent of untreated control values (arbitrary units).

FIGS. 2A-2F: Effect of Avicins on HSF1 Protein and Transcription of Stress Proteins. Translocation of the HSF1 transcription factor was examined by western blot analysis of cytoplasmic extracts (CE) and nuclear extracts (NE) prepared from Jurkat cells treated with avicin D for various time intervals. About 50 μg of the proteins were resolved on SDS-10% PAGE and probed with anti-HSF1 antibodies (FIG. 2A). FIG. 2B shows the densitometric analysis of the HSF1 protein in the CE fraction and the NE fraction. Total RNA from avicin D treated Jurkat cells was prepared as mentioned in Example 1 and used for one-step RT-PCR assay. Twenty PCR cycles were performed and the reaction products separated and viewed by ethidium bromide staining (FIG. 2C). FIG. 2D shows the densitometric analysis of the transcripts. FIG. 2E shows the northern blot analysis for Hsp70 and Hsp90. Staining the nylon membrane with methylene blue for 18S monitored the loading pattern. FIG. 2F shows the densitometric analysis of the northern blot. The values plotted in the graph are expressed as the percent change with respect to the value of the untreated cells.

FIG. 3: Post-Transcriptional Regulation of Hsp70 by Avicin D. Jurkat cells were treated for 2 hours and 4 hours with avicin D or pretreated with lactacystin (10 μM, 30 minutes) followed by treatment with avicin D for 4 hours. CE proteins (50 μg) were resolved on SDS PAGE, blotted, and probed with anti-Hsp70 and anti-Hsp90 antibodies. Loading of the proteins was examined by blotting the membranes, with β-actin antibodies.

FIGS. 4A-4C: Avicins Induce Ubiquitination. An in vitro ubiquitination assay using recombinant Hsp70, his-tagged ubiquitin, and CE proteins from avicin D treated cells was performed. His-tagged proteins were affinity purified and probed with anti-Hsp70 antibodies (FIG. 4A). Lane Un represents CE proteins from untreated cells. Lane L represents the control reaction where no CE proteins were used.

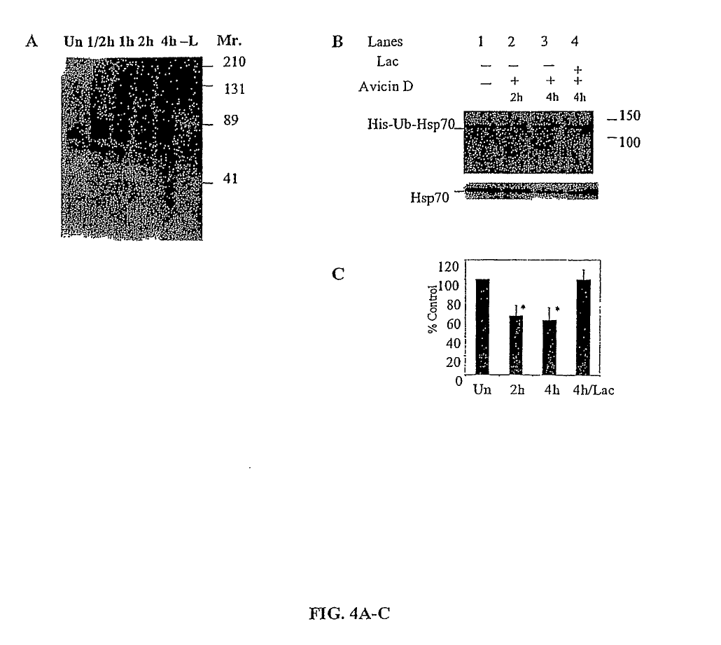

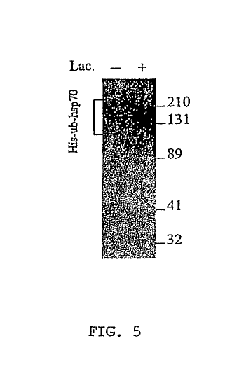

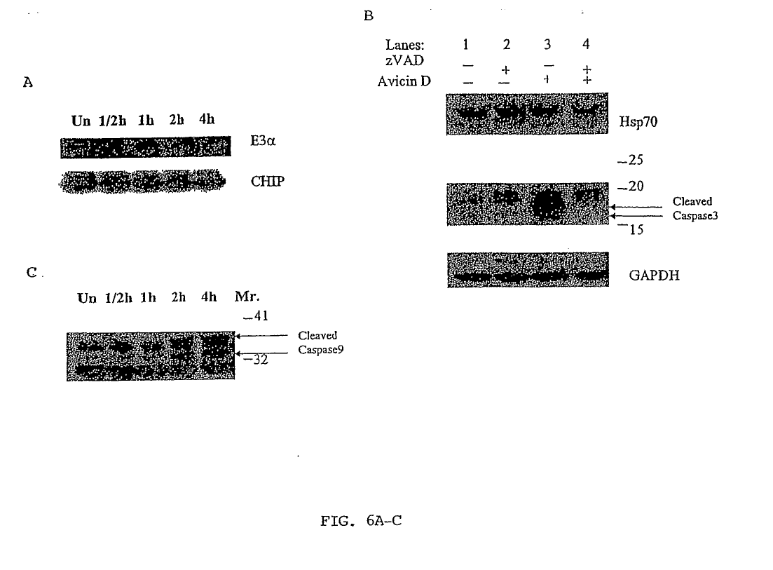

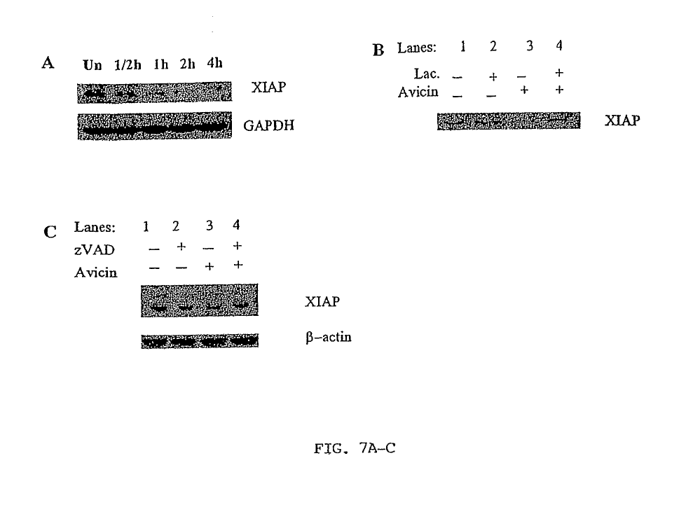

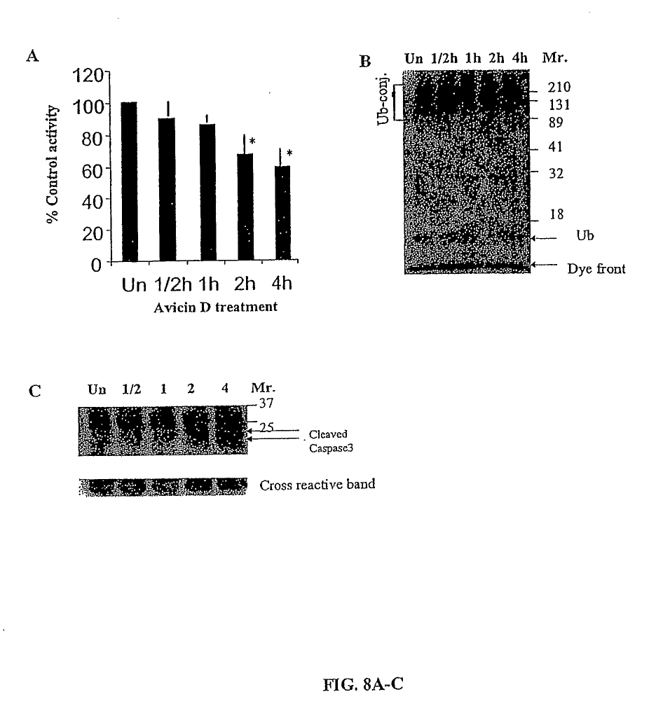

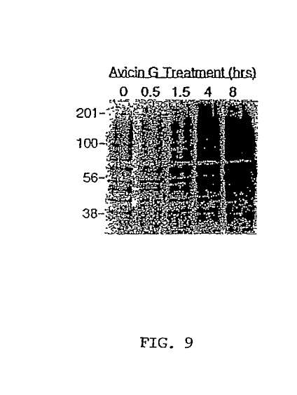

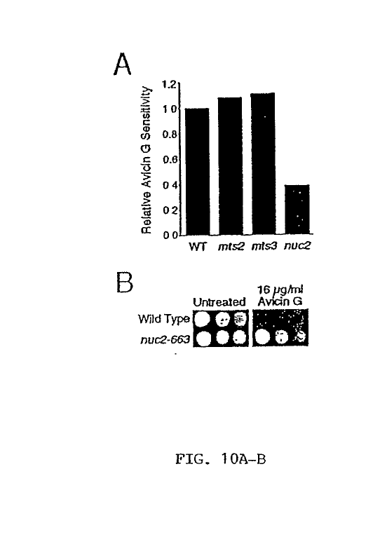

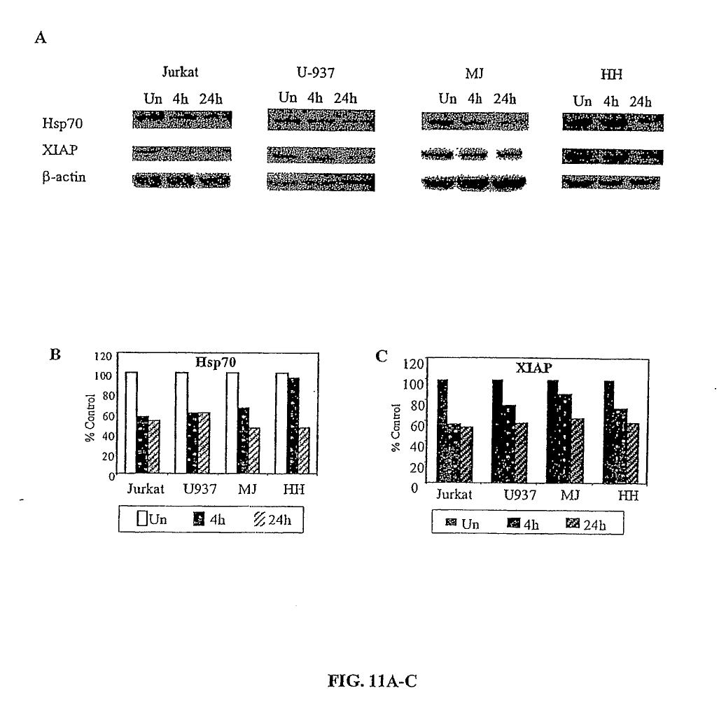

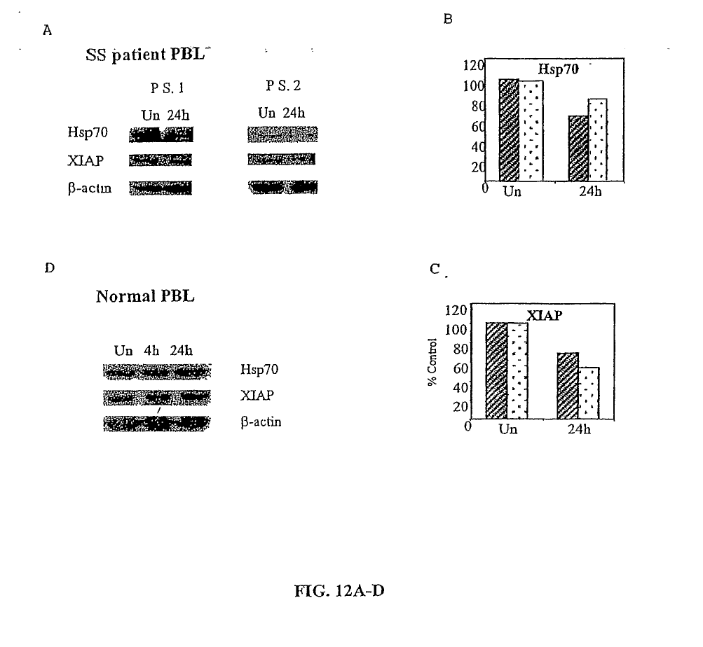

In vivo ubiquitination of Hsp70 was monitored by transfection of Jurkat cells with his-ub expressing plasmid that were treated with avicin D (1 μM) for 2 hours (FIG. 4B, lane 2) and 4 hours (FIG. 4B, lane 3), or pretreated with lactacystin (10 μM, 30 minutes) followed by avicin D for 4 hours (FIG. 4B, lane 4). His-tagged proteins were affinity purified and probed with anti-Hsp70 antibodies (FIG. 4B, upper panel). Total CE proteins (25 μg) from the same experiment were resolved on SDS-PAGE and probed with anti-Hsp70 antibodies (FIG. 4B, lower panel). The his-ub-Hsp70 protein band was quantitated by densitometry and expressed as percent change of untreated cells (FIG. 4C, *p<0.05 (Students t-test)).

FIG. 5: In Vivo Ubiquitination of Hsp70. Jurkat cells transfected with his-ub plasmid were treated with lactacystin (10 μM, 4 hours). During cell lysis, 0.2 mM NEM was added to the CE buffer to stabilize the his-ub-Hsp70 bands. His-tagged proteins were affinity purified from CE proteins and probed with anti-Hsp70 antibodies. Molecular weight is shown on the right.

FIGS. 6A-6C: Avicins Induce E3α Ubiquitin Ligase. FIG. 6A shows western analysis of CE proteins (50 μg) from avicin D treated Jurkat T cells probed with anti-E3α antibodies and with anti-CHIP antibodies. FIG. 6B shows Jurkat cells treated with zVAD-FMK (50 μM, lane 2) or avicin D (1 μM, 4 hours; lane 3) or pretreated with zVAD-FMK 30 minutes prior to avicin D treatment (lane 4). CE proteins were probed for Hsp70 (FIG. 6B, upper panel), caspase 3 (FIG. 6B, middle panel, the cleaved products of caspase 3 are marked with arrows) and GAPDH (FIG. 6B, lower panel). FIG. 6C represents the western analysis of CE proteins (50 μg) from avicin D treated Jurkat T cells probed with anti-caspase 9 antibodies. The cleaved products of caspase 9 are marked with arrows.

FIGS. 7A-7C: Role of E3α Ubiquitin Ligase in the Degradation of XIAP. FIG. 7A shows western blot analysis of CE proteins (50 μg) from avicin D treated cells probed with anti-XIAP antibodies. The blot was probed for GAPDH as a protein loading control. FIG. 7B shows western blot analysis of Jurkat cells treated with lactacystin (lane 2), avicin D (lane 3) or pretreated with lactacystin 30 minutes prior to avicin D for 4 hours (lane 4). CE proteins were probed with anti-XIAP antibodies. FIG. 7C shows western blot analysis of Jurkat cells treated with zVAD-FMK (50 μM), avicin D (lane 3) or pretreated with zVAD prior to avicin D treatment for 4 hours (lane 4). CE proteins were probed with anti-XIAP antibodies. β-actin was used as a protein loading control.

FIG. 8A-8C: Effect of Avicin D on Proteasomal Activity. Jurkat cells treated with avicin D were used to determine proteasomal activity. FIG. 8A shows the fluorescence measurement values obtained from three independent experiments and represented as percent control with respect to untreated cells. T-test significance shows *P<0.05. FIG. 8B shows western blot analysis of about 50 μg of CE proteins from Jurkat cells treated with avicin D separated on SDS-12.5% PAGE and probed with anti-ubiquitin antibodies to detect ubiquitin-protein conjugates. Ubiquitin and the dye-front are appropriately marked. FIG. 8C shows western blot analysis of CE proteins (50 μg) from Jurkat cells treated with avicin D for various time intervals, to examine caspase 3 activation. A protein band cross-reacting with caspase 3 antibody is shown to see the loading pattern.

FIG. 9: Avicin G Causes Hyperaccumulation of Ubiquitinated Proteins in S. pombe Cells. Wild-type S. pombe cells were incubated in YEAU containing 20 μg/ml avicin G for time indicated (hours), then processed for immunoblot analysis of ubiquitinated proteins. An increase in the levels of ubiquitinated proteins was detected after 1.5 hours of avicin G treatment.

FIGS. 10A and 10B: Effects of Avicin G on the Growth of S. pombe Mutants. Wild type, mts2-1 (mts2), mts3-1 (mts3), and nuc2-663 cells were spread on YEAU plates. Avicin G (25 jug) was then spotted onto the respective cell lawns and the plates were incubated at 26° C. for 5 days. The relative avicin G sensitivity of each strain, based on measurements of areas of avicin G-induced growth inhibition, was then determined, with wild-type cells being normalized to a value of 1 (FIG. 10A). Serial dilutions (1:5) of wild-type and nuc2-663 cells were spotted onto YEAU or YEAU containing 16 μg/ml avicin G and incubated for 5 days at 26° C. nuc2-663 cells, but not wild-type cells, grew on the avicin G plate (FIG. 10B).

FIGS. 11A-11C. Effect of Avicin D on Hsp70 and XIAP Proteins. Various cell-lines (Jurkat, U-937, MJ, and HH) were treated with avicin D for 4 and 24 hours. CE proteins were resolved on SDS-10% PAGE and probed with anti-Hsp70, anti-XIAP and anti-β-actin antibodies (FIG. 11A). The autoradiographic signals were quantified by densitometry and the values represented as percent control values of untreated cells (FIGS. 11B and 11C).

FIGS. 12A-12D. Effect of Avicin D on Hsp70 and XIAP Proteins in Primary PBL Cells. PBL cells from two SS patients (P.S.1 and P.S.2) were treated with avicin D. CE proteins were probed with anti-Hsp70, anti-XIAP and anti-β-actin antibodies (FIG. 12A). The autoradiographic signals were quantified by densitometry and the values represented as percent control values of untreated cells (FIGS. 12B and 12C). Normal PBL cells were treated with avicin D and CE proteins probed with anti- Hsp70, anti-XIAP, and anti-#-actin antibodies (FIG. 12D).

### DETAILED DESCRIPTION OF THE INVENTION

Triterpenoid compounds affect multiple cellular processes. For example, perturbation of the mitochondria by triterpenoid compounds has been shown to initiate the apoptotic response (Haridas et al., 2001). In addition, triterpenoid compounds have been shown to inhibit inflammation by redox regulation of transcription factors (Haridas et al., 2001; Haridas et al., 2004). The inventors have now demonstrated the activation of the ubiquitin pathway by triterpenoid compounds removes post-mitochondrial barriers to apoptosis. In particular, the inventors demonstrated that Hsp70 is polyubiquitinated prior to down-regulation of the protein, and that triterpenes enhance auto-ubiquitination and degradation of XIAP by the ring finger E3α/degron pathway. The ability of triterpenes to induce ubiquitination and regulate the degradation of Hsp70 and XIAP has important implications in the treatment of malignancies and inflammatory disorders. Based on these observations, the inventors developed novel methods and compositions for the treatment of malignancies and inflammatory disorders that employ triterpene compounds in combination with proteasome inhibitors.

Drugs that inhibit the proteasome have shown promising results as anti-cancer agents (Hideshima et al., 2001; Mitsiades et al., 2002). Although triterpenes, such as avicins, share some properties of proteasome inhibitors, significant differences exist. For example, proteasome inhibitors like PS341 (Velcade®) generally suppress 20S activity completely, whereas avicins only partially suppress 20S activity. Both compounds suppress NF-kB, but avicins do so by redox regulation (Haridas et al., 2001). Both PS341 (Mitsiades et al., 2002) and avicins (Mujoo et al., 2001) inhibit the PI3K/Akt pathway. However, in contrast to avicins, the proteasome inhibitors potently activate stress responses and up-regulate expression of Hsp70 and Hsp90 (42).

### A. TRITERPENOIDS

Triterpenoids form the largest and most diverse class of organic compounds found in plants (Mahato & Sen, 1997). They exhibit enormous chemical variety and complexity but have a common biosynthetic origin, the fusion of five-carbon units, each having an isoprenoid structure (Wendt et al., 2000). Methods for isolating, characterizing, modifying, and using triterpenoid compounds can be found in U.S. Pat. No. 6,444,233, which is incorporated in its entirety by reference.

Triterpene saponins particularly have been the subject of much interest because of their biological properties. Pharmacological and biological properties of triterpene saponins from different plant species have been studied, including fungicidal, anti-viral, anti-mutagenic, spermicidal or contraceptive, cardiovascular, and anti-inflammatory activities (Hostettmann et al., 1995).

Avicins are triterpenoid electrophilic metabolite molecules isolated from an Australian desert plant, Acacia victoriae. A series of studies have identified cancer and inflammatory diseases as potential clinical targets for avicins (Haridas et al., 2001; Haridas et al., 2001; Haridas et al., 2004; Hanausek et al., 2001; Mujoo et al., 2001; Jayatilake et al., 2003). There is evidence that avicins induce stress resistance in human cells in a redox dependent manner, and that their pro-apoptotic property appears to be independent of p53.

The inventors have further elucidated the molecular mechanisms by which avicins inhibit tumor cell growth and modulate inflammation by demonstrating that avicins can regulate post-mitotic events in apoptosis through their ability to down-regulate the anti-apoptotic proteins Hsp70 and Hsp 90, as well as XIAP. The inventors showed avicin-mediated degradation of Hsp70 and XIAP via activation of the ubiquitin/proteasomal pathway. From these observations, the inventors propose that avicins regulate a highly coordinated programmed response to stress, in which transcription factors are regulated by redox-modification to maintain homeostatic balance and other proteins are removed to enhance destruction of damaged cells. The overall effect is to shift energy requirements from immediate needs to that associated with repair or maintenance of somatic health. Thus, a rapid and selective regulation of stress by the avicins acts as a molecular switch to control cell death and life, inflammation, and other aspects of metabolism.

Based on these observations, the inventors developed novel methods and compositions for the treatment of malignancies and inflammatory disorders that employ natural triterpene compounds in combination with proteasome inhibitors. Recently, a synthetic triterpene (CDDO-Im) has been shown to be synergistic with PS341 in triggering apoptosis in multiple myeloma (MM) cells (Chauhan et al., 2004).

Other triterpenoids that exhibit pharmacological properties include glycyrrhetinic acid, and certain derivatives thereof, which are known to have anti-ulcer, anti-inflammatory, anti-allergic, anti-hepatitis and antiviral actions. For instance, certain glycyrrhetinic acid derivatives can prevent or heal gastric ulcers (Doll et al., 1962). Among such compounds known in the art are carbenoxolone (U.S. Pat. No. 3,070,623), glycyrrhetinic acid ester derivatives having substituents at the 3° position (U.S. Pat. No. 3,070,624), amino acid salts of glycyrrhetinic acid (Japanese Patent Publication JP-A-44-32798), amide derivatives of glycyrrhetinic acid (Belgian Patent No. 753773), and amide derivatives of 11-deoxoglycyrrhetinic acid (British Patent No. 1346871). Glycyrrhetinic acid has been shown to inhibit enzymes involved in leukotriene biosynthesis, including 5-lipoxygenase activity, and this is thought to be responsible for the reported anti-inflammatory activity (Inoue et al., 1986).

Betulinic acid, a pentacyclic triterpene, is reported to be a selective inhibitor of human melanoma tumor growth in nude mouse xenograft models and was shown to cause cytotoxicity by inducing apoptosis (Pisha et al., 1995). A triterpene saponin from a Chinese medicinal plant in the Cucurbitaceae family has demonstrated anti-tumor activity (Kong et al., 1993). Monoglycosides of triterpenes have been shown to exhibit potent and selective cytotoxicity against MOLT-4 human leukemia cells (Kasiwada et al., 1992) and certain triterpene glycosides of the Iridaceae family inhibited the growth of tumors and increased the life span of mice implanted with Ehrlich ascites carcinoma (Nagamoto et al., 1988). A saponin preparation from the plant Dolichos falcatus, which belongs to the Leguminosae family, has been reported to be effective against sarcoma-37 cells in vitro and in vivo (Huang et al., 1982). Soya saponin, also from the Leguminosae family, has been shown to be effective against a number of tumors (Tomas-Barbaren et al., 1988). Some triterpene aglycones also have been demonstrated to have cytotoxic or cytostatic properties, i.e., stem bark from the plant Crossopteryx febrifuga (Rubiaceae) was shown to be cytostatic against Co-115 human colon carcinoma cell line in the ng/ml range (Tomas-Barbaren et al., 1988).

### B. THE UBIQUITIN/PROTEASOME PATHWAY

As mentioned above, the inventors have demonstrated the ability of triterpenoid compounds to induce the ubiquitination and degradation of anti-apoptotic proteins. The ubiquitin/proteasome pathway is the major proteolytic system in the cytosol and nucleus of eukaryotic cells. The majority of substrates of the pathway are marked for degradation by covalent attachment of multiple ubiquitin molecules. Ubiquitination involves three steps that utilize E1 (activating enzyme), E2 (conjugating enzyme), and E3 ligases. E3 ligases play a central regulatory role in that they provide substrate specificity to the ubiquitin/proteasome pathway.

The ubiquitin/proteasome pathway is responsible for the breakdown of a large variety of cell proteins and is essential for many cellular regulatory mechanisms. For example, cell cycle progression is controlled by the proteasomal degradation of cyclins and inhibitors of cyclin-dependent kinases (Koepp et al., 1999), while degradation of transcriptional regulators, such as c-Jun, E2F-1, and β-catenin, is essential for the regulation of cell growth and gene expression (Hershko et al., 1998). In addition, proteasomal degradation of the IκB inhibitor of the transcription factor NF-κB is essential for the development of inflammatory response (Meng et al., 1999; Palombella et al., 1998).

The ubiquitin/proteasome pathway has been proposed to play a key role in the regulation of apoptosis. Degradation of the tumor suppressor p53, and p27^{Kip1} inhibitor of cyclin-dependent kinases by the ubiquitin/proteasome pathway has been shown to promote tumorigenesis (Hershko et al., 1998; Pagano et al., 1995). Specific inhibitors of the proteasome have been shown to induce apoptosis by accumulation of pro-apoptotic molecules and other less characterized mechanisms (Jesenberger and Jentsch, 2002). In addition, proteasome inhibitors have been shown to reduce inflammation As will be discussed in more detail below, proteasome inhibitors and triterpenoid compounds can be used in combination to provide novel treatments for cancer and inflammatory disorders.

### C. PROTEASOME INHIBITORS

Inhibitors of the proteasome block the degradation of many cellular proteins. Although the proteasome has multiple active sites, inhibition of all of them is not required to significantly reduce protein degradation. Major classes of proteasome inhibitors include peptide benzamides, peptide α-ketoamides, peptide aldehydes, peptide α-ketoaldehydes, peptide vinyl sulfones, peptide boronic acids, linear peptide epoxyketones, peptide macrocycles, γ-lactam thiol ester, and epipolythiodioxopiperazine toxin.

Proteasome inhibitors are usually short peptides linked to a pharmacore. Specific examples of proteasome inhibitors include MG132, boronate MG132, MG262, boronate MG262, MG115, ALLN, PSI, CEP1612, epoxomicin, eponemycin, epoxyketone eponemycin, dihydroeponemycin, LLM, PS-341, DFLB, PS-273, ZLVS, NLVS, and TMC-95A. Examples of non-peptide proteasome inhibitors include lactacystin, β-lactone, gliotoxin, EGCG). Additional examples of proteasome inhibitors are disclosed in Kisselev and Goldberg (2001) and Myung et al. (2001), both of which are incorporated herein in their entirety.

The ability of proteasome inhibitors to inhibit cell proliferation, induce apoptosis, and inhibit angiogenesis makes these compounds attractive candidates for anti-cancer drugs. However, inhibition of proteasomal function is a potent stimulus of the heat shock protein response, likely due to the accumulation of undegraded proteins (Lee and Goldberg, 1998). As mentioned above, acquired resistance to apoptosis is a hallmark of most types of cancer, and overexpression of heat shock proteins is a prominent mechanism of acquired resistance to apoptosis. The inventors discovery that triterpene compounds can downregulate heat shock proteins, as well as the anti-apoptotic XIAP protein, led to the development of a novel method for treating malignant disease using a triterpene compound in combination with a proteasome inhibitor.

### D. HEAT SHOCK PROTEINS

The inventors' elucidation of the regulation of specific heat shock proteins by triterpenoid compounds provides a novel approach to cancer therapy and the regulation of inflammation. Heat shock proteins are a family of proteins that protect a cell against environmental stressors. Under conditions of stress such as heat, exposure to heavy metals, and toxins, ischemia/reperfusion injury, or oxidative stress from inflammation, Hsp induction is both rapid and robust. Induction of heat shock proteins by a mild “stress” confers protection against subsequent insult or injury, which would otherwise lead to cell death. Expression of inducible heat shock proteins is known to correlate with increased resistance to apoptosis induced by a range of diverse cytotoxic agents and has been implicated in chemotherapeutic resistance of tumors and carcinogenesis(Creagh et al., 2000).

The inventors demonstrated the ability of a triterpenoid to down-regulate anti-apoptotic proteins Hsp70 and Hsp90. Hsp70 is overexpressed in many malignancies. It inhibits key effectors of the apoptotic machinery including the apoptosome, the caspase activation complex, and apoptosis inducing factor. In addition, it plays a role in the proteasome-mediated degradation of apoptosis-regulatory proteins. Hsp90 is overexpressed in many malignancies, and is required for the conformational stability and function of a wide range of oncogenic proteins, including c-Raf-1, Cdk4, ErbB2, mutant p53, c-Met, Polo-1 and telomerase hTERT.

Hsps are regulated at the transcriptional level by the heat shock factor (HSF1), which under stressed conditions resides in the cytoplasm as an inactive monomer. Under stress, HSF1 undergoes oligomerization and nuclear translocation prior to the transcription of Hsp genes. However, the inventors showed that the triterpenoid-induced decrease in Hsp70 and Hsp90 was not at the level of transcription. Rather, it was shown that the triterpenoid induced the ubiquitination and subsequent proteolytic degradation of Hsp70. This observation elucidates a novel mechanism for regulating a chaperone protein via enhanced ubiquitination.

Methods of analyzing the expression of inducible heat shock proteins are known to those of skill in the art. For example, heat shock proteins can be assayed by standard western blot analysis using monoclonal antibodies to the specific isoforms. Immunoblots for the constitutive heat shock cognates, such as hsp60 and hsc70, can be performed to check the specificity of response and insure equal loading of lanes (the expression of these proteins usually remains constant). In addition, antibodies can be used to detect the expression of heat shock proteins by immunofluorescence and ELISA.

The expression of heat shock proteins can also be evaluated at the transcription level by a variety of methods known to those of skill in the art. For example, Hsp mRNA levels can be assayed using RT-PCR, genomic microarrays, or real-time PCR. Another approach for analyzing the expression of heat shock proteins is the use of electrophoretic mobility shift assays to look at binding of the transcription factor HSF-1. In addition, HSE-luciferase reporter assays can be employed to measure activity of the transcription factor HSF-1.

### E. X-LINKED INHIBITOR OF APOPTOSIS PROTEIN

X-linked inhibitor of apoptosis protein (XIAP), a member of the IAP (Inhibitor of Apoptosis Proteins) gene family, is a potent anti-apoptotic factor. XIAP inhibits apoptosis by binding to and blocking the action of several different caspases. XIAP is known to block caspase-3, caspase-7, and caspase-9. XIAP is frequently overexpressed in cancer cells, and is associated with poor clinical outcome (Yang and Yu, 2003; Holcik et al., 2001). Recently, it was reported that a small molecule antagonist of XIAP may overcome resistance to apoptosis in tumor cells (Schimmer et al., 2004).

The inventors demonstrated a significant decrease in XIAP protein in cells treated with triterpenoid compounds. It was also shown that lactacystin blocked the triterpene-induced decrease in XIAP protein, confirming a proteasome-based degradation of XIAP. In addition, avicin-induced XIAP degradation was partially blocked by the caspase inhibitor zVAD-fmk. These results indicate that triterpenes enhance both auto-ubiquitination, as well as degradation of XIAP by the ring finger E3α/degron pathway. The inventors propose that the regulation of XIAP together with heat shock proteins will offer a new approach to cancer therapy.

Methods of analyzing XIAP expression are known to those of skill in the art. For example, XIAP protein can be assayed by standard western blot analysis. In addition, antibodies can be used to detect XIAP by immunofluorescence and ELISA. Other methods of analyzing XIAP expression include assaying XIAP mRNA levels using, for example, RT-PCR, genomic microarrays, and real-time PCR. Furthermore, the interaction of XIAP with caspases can be assessed by binding assays known to those of skill in the art. Caspase activity can also be assessed using enzyme assays, such as those described in Suzuki et al., (2001).

### F. TREATMENT OF CANCER AND INFLAMMATION WITH THE TRITERPENE COMPOUNDS AND PROTEASOME INHIBITORS

Based on the observation that triterpenes can mediated the degradation of Hsp70 and XIAP via activation of the ubiquitin/proteasome pathway, the inventors developed novel approaches to the treatment of cancer and inflammation. The present invention provides methods for treating malignancies and inflammation comprising administering to a subject a triterpene compound and a proteasome inhibitor. Proteasome inhibitors suppress the activity of the proteasome, and have shown promise as anti-cancer agents. However, proteasome inhibitors potently activate stress responses and upregulate the expression of inducible heat shock proteins. As demonstrated by the inventors, levels of anti-apoptotic proteins Hsp70, Hsp90, and XIAP are decreased in triterpene-treated cells. Therefore, triterpenes can be used synergistically with proteasome inhibitors. Given the role of Hsps and the proteasome in inflammation and cancer, the present invention would be useful in the treatment and prevention of both inflammatory disorders and cancer, particularly drug-resistant cancers.

A subject may be treated prophylactically to prevent cancer or inflammation or therapeutically after the cancer or an inflammatory disorder has begun. To kill cells, inhibit cell growth, inhibit metastasis, decrease tumor size and otherwise reverse or reduce the malignant phenotype of tumor cells, using the methods and compositions of the present invention, one would generally contact a “target” cell with a triterpene compound and a proteasome inhibitor as described herein. This may be achieved by contacting a tumor or tumor cell with a single composition or pharmacological formulation that includes the triterpene compound and the proteasome inhibitor or by contacting a tumor or tumor cell with more than one distinct composition or formulation, at the same time, wherein one composition includes the triterpene compound and the other includes the proteasome inhibitor.

Cancer cells for treatment with the instant invention include ovarian, pancreatic, leukemia, breast, melanoma, prostate, lung, brain, kidney, liver, skin, stomach, esophagus, head and neck, testicles, colon, cervix, lymphatic system, larynx, esophagus, parotid, biliary tract, rectum, uterus, endometrium, kidney, bladder, and thyroid; including squamous cell carcinomas, adenocarcinomas, small cell carcinomas, gliomas, neuroblastomas, and the like. However, this list is for illustrative purposes only, and is not limiting, as potentially any tumor cell could be treated with the compounds of the instant invention. Assay methods for ascertaining the relative efficacy of the compounds of the invention in treating the above types of tumor cells and other tumor cells are specifically disclosed herein and will be apparent to those of skill in the art in light of the present disclosure.

The invention compounds are preferably administered as a pharmaceutical composition comprising a pharmaceutically or pharmacologically acceptable diluent or carrier. The nature of the carrier is dependent on the chemical properties of the compounds, including solubility properties, and/or the mode of administration. For example, if oral administration is desired, a solid carrier may be selected, and for i.v. administration a liquid salt solution carrier may be used.

The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

(i) Parenteral Administration

One embodiment of the invention provides formulations for parenteral administration, e.g., formulated for injection via the intravenous, intramuscular, subcutaneous or other such routes, including direct instillation into a tumor or disease site. The preparation of an aqueous compositions that contains a triterpene compound and a proteasome inhibitor will be known to those of skill in the art in light of the present disclosure. Typically, such compositions can be prepared as injectables, either as liquid solutions or suspensions; solid forms suitable for using to prepare solutions or suspensions upon the addition of a liquid prior to injection also can be prepared; and the preparations also can be emulsified.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

The triterpene compounds and proteasome inhibitors can be formulated into a composition in a neutral or salt form. Pharmaceutically acceptable salts include the acid addition salts, which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

(ii) Other Modes of Administration

Other modes of administration will also find use with the subject invention. For instance, the triterpene compounds and proteasome inhibitors of the invention may be formulated in suppositories and, in some cases, aerosol and intranasal compositions. For suppositories, the vehicle composition will include traditional binders and carriers such as polyalkylene glycols or triglycerides. Such suppositories may be formed from mixtures containing the active ingredient in the range of about 0.5% to about 10% (w/w), preferably about 1% to about 2%.

Oral compositions may be prepared in the form of solutions, suspensions, tablets, pills, capsules, sustained release formulations, or powders. These compositions can be administered, for example, by swallowing or inhaling. Where a pharmaceutical composition is to be inhaled, the composition will preferably comprise an aerosol. Exemplary procedures for the preparation of aqueous aerosols for use with the current invention may be found in U.S. Pat. No. 5,049,388, the disclosure of which is specifically incorporated herein by reference in its entirety. Preparation of dry aerosol preparations are described in, for example, U.S. Pat. No. 5,607,915, the disclosure of which is specifically incorporated herein by reference in its entirety.

Also useful is the administration of the invention compounds directly in transdermal formulations with permeation enhancers such as DMSO. These compositions can similarly include any other suitable carriers, excipients or diluents. Other topical formulations can be administered to treat certain disease indications. For example, intranasal formulations may be prepared which include vehicles that neither cause irritation to the nasal mucosa nor significantly disturb ciliary function. Diluents such as water, aqueous saline or other known substances can be employed with the subject invention. The nasal formulations also may contain preservatives such as, but not limited to, chlorobutanol and benzalkonium chloride. A surfactant may be present to enhance absorption of the subject compounds by the nasal mucosa.

(iii) Formulations and Treatments

Upon formulation, solutions will be administered in a manner compatible with the dosage formulation and in such amount as is therapeutically effective. The formulation of choice can be accomplished using a variety of excipients including, for example, pharmaceutical grades of mannitol, lactose, starch, magnesium stearate, sodium saccharin cellulose, magnesium carbonate, and the like.

Typically, the compounds of the instant invention will contain from less than 1% to about 95% of the active ingredient, preferably about 10% to about 50%. Preferably, between about 10 mg/kg patient body weight per day and about 25 mg/kg patient body weight per day will be administered to a patient. The frequency of administration will be determined by the care given based on patient responsiveness. Other effective dosages can be readily determined by one of ordinary skill in the art through routine trials establishing dose response curves.

Regardless of the mode of administration, suitable pharmaceutical compositions in accordance with the invention will generally include an amount of the triterpene compound and the proteasome inhibitor admixed with an acceptable pharmaceutical diluent or excipient, such as a sterile aqueous solution, to give a range of final concentrations, depending on the intended use. The triterpenoid compound and the proteasome inhibitor may be prepared in a single pharmaceutical composition or in separate pharmaceutical compositions. The techniques of preparation are generally well known in the art as exemplified by Remington's Pharmaceutical Sciences, 16th Ed. Mack Publishing Company, 1980, which reference is specifically incorporated herein by reference in its entirety. For human administration, preparations should meet sterility, pyrogenicity, general safety and purity standards as required by FDA Office of Biological Standards.

The therapeutically effective doses are readily determinable using an animal model, as shown in the studies detailed herein. For example, experimental animals bearing solid tumors are frequently used to optimize appropriate therapeutic doses prior to translating to a clinical environment. Such models are known to be very reliable in predicting effective anti-cancer strategies. Likewise, animal models for inflammatory disorder are known in the art and may be used to optimize appropriate therapeutic doses prior to translating to a clinical environment.

In certain embodiments, it may be desirable to provide a continuous supply of therapeutic compositions to the patient. For intravenous or intraarterial routes, this is accomplished by drip system. For topical applications, repeated application would be employed. For various approaches, delayed release formulations could be used that provided limited but constant amounts of the therapeutic agent over and extended period of time. For internal application, continuous perfusion of the region of interest may be preferred. This could be accomplished by catheterization, post-operatively in some cases, followed by continuous administration of the therapeutic agent. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the injections are administered. It is believed that higher doses may be achieved via perfusion, however.

### 1. Treatment of Artificial and Natural Body Cavities

One of the prime sources of recurrent cancer is the residual, microscopic disease that remains at the primary tumor site, as well as locally and regionally, following tumor excision. In addition, there are analogous situations where natural body cavities are seeded by microscopic tumor cells. The effective treatment of such microscopic disease would present a significant advance in therapeutic regimens.

Thus, in certain embodiments, a cancer may be removed by surgical excision, creating a “cavity.” Both at the time of surgery, and thereafter (periodically or continuously), the therapeutic composition of the present invention is administered to the body cavity. This is, in essence, a “topical” treatment of the surface of the cavity. The volume of the composition should be sufficient to ensure that the entire surface of the cavity is contacted by the expression construct.

In one embodiment, administration simply will entail injection of the therapeutic composition into the cavity formed by the tumor excision. In another embodiment, mechanical application via a sponge, swab or other device may be desired. Either of these approaches can be used subsequent to the tumor removal as well as during the initial surgery. In still another embodiment, a catheter is inserted into the cavity prior to closure of the surgical entry site. The cavity may then be continuously perfused for a desired period of time.

In another form of this treatment, the “topical” application of the therapeutic composition is targeted at a natural body cavity such as the mouth, pharynx, esophagus, larynx, trachea, pleural cavity, peritoneal cavity, or hollow organ cavities including the bladder, colon or other visceral organs. In this situation, there may or may not be a significant, primary tumor in the cavity. The treatment targets microscopic disease in the cavity, but incidentally may also affect a primary tumor mass if it has not been previously removed or a pre-neoplastic lesion which may be present within this cavity. Again, a variety of methods may be employed to affect the “topical” application into these visceral organs or cavity surfaces. For example, the oral cavity in the pharynx may be affected by simply oral swishing and gargling with solutions. However, topical treatment within the larynx and trachea may require endoscopic visualization and topical delivery of the therapeutic composition. Visceral organs such as the bladder or colonic mucosa may require indwelling catheters with infusion or again direct visualization with a cystoscope or other endoscopic instrument. Cavities such as the pleural and peritoneal cavities may be accessed by indwelling catheters or surgical approaches which provide access to those areas.

Many inflammatory diseases will also be amenable to the “topical” application of the therapeutic composition to a natural body cavity such as the mouth, pharynx, esophagus, larynx, trachea, pleural cavity, peritoneal cavity, or hollow organ cavities including the bladder, colon or other visceral organs. For example, topical application to the intestinal epithelium may be used in the treatment of inflammatory bowel disorders, such as Crohn's disease and ulcerative colitis. As another example, topical application to the bladder could be useful for the treatment of diseases, such as interstitial cystitis. Again, a variety of methods may be employed to affect the “topical” application into these visceral organs or cavity surfaces. Visceral organs, such as the bladder or colonic mucosa, may require indwelling catheters with infusion or direct visualization with a cystoscope or other endoscopic instrument. Cavities such as the pleural and peritoneal cavities may be accessed by indwelling catheters or surgical approaches which provide access to those areas.

### 2. Prevention of Cancer with the Compounds of the Invention

Another application of the compounds of the invention is in the prevention of cancer in high risk groups. Such patients (for example, those with genetically defined predisposition to tumors such as breast cancer, colon cancer, skin cancer, and others) would be treated by mouth (gastrointestinal tumors), topically on the skin (cutaneous), or by systemic administration for a minimum period of one year and perhaps longer to determine prevention of cancer. This use would include patients and well defined pre-neoplastic lesions, such as colorectal polyps or other premalignant lesions of the skin, breast, lung, or other organs.

(iv) Therapeutic Kits

The present invention also provides therapeutic kits comprising the compositions described herein. Such kits will generally contain, in suitable container means, a pharmaceutically acceptable formulation of at least one triterpene compound and at least one proteasome inhibitor in accordance with the invention. The kits also may contain other pharmaceutically acceptable formulations, such as those containing components to target the triterpene compound to distinct regions of a patient where treatment is needed, or any one or more of a range of drugs which may work in concert with the triterpene compounds and the proteasome inhibitors, for example, chemotherapeutic agents.

The kits may have a single container means that contains the triterpene compounds and the proteasome inhibitors, with or without any additional components, or they may have distinct container means for each desired agent. When the components of the kit are provided in one or more liquid solutions, the liquid solution is an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent also may be provided in another container means. The container means of the kit will generally include at least one vial, test tube, flask, bottle, syringe or other container means, into which the desired agents may be placed and, preferably, suitably aliquoted. Where additional components are included, the kit will also generally contain a second vial or other container into which these are placed, enabling the administration of separately designed doses. The kits also may comprise a second/third container means for containing a sterile, pharmaceutically acceptable buffer or other diluent.

The kits also may contain a means by which to administer the therapeutic compositions to an animal or patient, e.g., one or more needles or syringes, or even an eye dropper, pipette, or other such like apparatus, from which the formulation may be injected into the animal or applied to a diseased area of the body. The kits of the present invention will also typically include a means for containing the vials, or such like, and other component, in close confinement for commercial sale, such as, e.g., cardboard containers or injection or blow-molded plastic containers into which the desired vials and other apparatus are placed and retained.

### G. TREATMENT WITH ADDITIONAL THERAPEUTIC AGENTS

In certain embodiments of the present invention, it may be desirable to administer the triterpene compounds and proteasome inhibitors of the invention in combination with one or more other agents having anti-tumor activity or anti-inflammatory activity. This may enhance the overall anti-tumor or anti-inflammatory activity achieved by therapy with the compounds of the invention alone. To use the present invention in combination with the administration of additional therapeutic agents, one would simply administer to an animal a triterpene compound and a proteasome inhibitor in combination with an additional therapeutic agent in a manner effective to result in their combined anti-tumor or anti-inflammatory actions within the animal. These agents would, therefore, be provided in an amount effective and for a period of time effective to result in their combined actions at the site of the tumor or inflammation. To achieve this goal, the therapeutic agents may be administered to the animal simultaneously, either in a single composition or as distinct compositions using different administration routes.

Alternatively, treatment with the triterpene compounds and the proteasome inhibitors may precede or follow treatment with the additional therapeutic agent by intervals ranging from minutes to weeks. In embodiments where an additional agent, the triterpene compound, and the proteasome inhibitor are administered separately to the animal, one would generally ensure that a significant period of time did not expire between the time of each delivery, such that the additional agent, the triterpene compound, and the proteasome inhibitor would still be able to exert an advantageously combined effect on the tumor or inflammation. In such instances, it is contemplated that one would contact the tumor or the site of inflammation with the therapeutic agents within about 5 minutes to about one week of each other and, more preferably, within about 12-72 hours of each other, with a delay time of only about 24-48 hours being most preferred. In some situations, it may be desirable to extend the time period for treatment significantly, where several days (2, 3, 4, 5, 6 or 7) or even several weeks (1, 2, 3, 4, 5, 6, 7 or 8) lapse between the respective administrations. It also is conceivable that more than one administration of one or more of the therapeutic agents will be desired. To achieve tumor regression or reduce inflammation, the therapeutic agents are delivered in a combined amount effective to inhibit tumor growth or reduce inflammation, irrespective of the times for administration.

A variety of agents are suitable for use in the combined treatment methods disclosed herein. Additional therapeutic agents that may be useful in the treatment of cancer include, for example, chemotherapeutics, radiation, and therapeutic proteins or genes. Chemotherapeutic agents contemplated as exemplary include, e.g., etoposide (VP-16), adriamycin, 5-fluorouracil (5-FU), camptothecin, actinomycin-D, mitomycin C, and cisplatin (CDDP). As will be understood by those of ordinary skill in the art, the appropriate doses of chemotherapeutic agents will be generally around those already employed in clinical therapies wherein the chemotherapeutics are administered alone or in combination with other chemotherapeutics. Further useful agents for the treatment of cancer include compounds that interfere with DNA replication, mitosis and chromosomal segregation. Such chemotherapeutic compounds include adriamycin, also known as doxorubicin, etoposide, verapamil, podophyllotoxin, and the like. The skilled artisan is directed to “Remington's Pharmaceutical Sciences” 15th Edition, chapter 33, in particular pages 624-652 for additional information in this regard. Some variation in dosage will necessarily occur depending on the condition of the subject being treated. The person responsible for administration will, in any event, determine the appropriate dose for the individual subject.

Other factors that cause DNA damage and have been used extensively include what are commonly known as γ-rays, X-rays, and/or the directed delivery of radioisotopes to tumor cells. Other forms of DNA damaging factors also are contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Additional therapeutic agents useful in the treatment of inflammation include aminosalicylates drugs, such as those that contain 5-aminosalicyclic acid (5-ASA), corticosteroids, such as prednisone and hydrocortisone, and immunomodulators, such as azathioprine and 6-mercapto-purine (6-MP).

### H. ASSAYS AND METHODS FOR SCREENING ACTIVE COMPOUNDS

A number of assays are known to those of skill in the art and may be used to further characterize the compositions of the invention. These include assays of biological activities as well as assays of chemical properties. The results of these assays provide important inferences as to the properties of compounds as well as their potential applications in treating human or other mammalian patients. Of particular interest are assays of specific combinations of natural triterpenoids and proteasome inhibitors. Assays deemed to be of particular utility include in vivo and in vitro screens of biological activity and immunoassays.

(i) In Vitro Assays

In one embodiment of the invention, screening of combinations of triterpenoid compounds and proteasome inhibitors is done in vitro to identify those combinations capable of inhibiting the growth of or killing tumor cells or reducing inflammation. Killing of tumor cells, or cytotoxicity, is generally exhibited by necrosis or apoptosis. Necrosis is a relatively common pathway triggered by external signals. During this process, the integrity of the cellular membrane and cellular compartments is lost. On the other hand, apoptosis, or programmed cell death, is a highly organized process of morphological events that is synchronized by the activation and deactivation of specific genes (Thompson et al., 1992; Wyllie, 1985).

Those of skill in the art will be familiar with a variety of in vitro assays to evaluate the impact of combinations of triterpenoid compounds and proteasome inhibitors on inflammation. For example, the induction of heat shock proteins can be assayed by standard western blot analysis using monoclonal antibodies to the specific isoforms. In addition, antibodies can be used to detect the expression of heat shock proteins by immunofluorescence and ELISA. Other methods of analyzing the induction of heat shock proteins include assaying hsp mRNA levels using, for example, RT-PCR, genomic microarrays, and real-time PCR. Another approach for analyzing the induction of heat shock proteins is the use of electrophoretic mobility shift assays to look at binding of the transcription factor HSF-1. In addition, HSE-luciferase reporter assays can be employed to measure activity of the transcription factor HSF-1.

The inhibition of the NF-κB pathway can also be assayed to evaluate the impact of combinations of triterpenoid compounds and proteasome inhibitors on inflammation. For example, electrophoretic mobility shift assays (EMSA or gel shifts) using an oligonucleotide labeled with ³²P can be performed to determine activation of NF-κB. Activation of NF-κB and release from the inhibitor IκB results in binding to this mimic, which can be easily detected on acrylamide gels. Two additional measures may be used to corroborate NF-κB activation. First, activated NF-κB translocates into the nucleus of the cell and therefore detection of NF-κB in the nucleus by immunofluorescence or immunoblotting of nuclear fractions strongly supports NF-κB activation. Second, transient transfections with a NF-κB sensitive reporter construct, which has five copies of the NF-κB responsive promoter element cloned in front of a firefly luciferase reporter, can be performed. ELISA-based assays for the detection of NF-κB activation are also known in the art. For example, an NF-κB ELISA-based assay kit is commercially available from Vinci-Biochem (Vinci, Italy).

Furthermore, NF-κB regulates a wide variety of genes encoding, for example, cytokines, cytokine receptors, cell adhesion molecules, proteins involved in coagulation, and proteins involved in cell growth. Thus, another approach to the study of the NF-κB pathway is through the analysis of the expression of genes known to be regulated by NF-κB. Those of skill in the art will be familiar with a variety of techniques for the analysis of gene expression. For example, changes in mRNA and/or protein levels may be measured. Changes in mRNA levels can be detected by numerous methods including, but not limited to, real-time PCR and genomic microarrays. Changes in protein levels may be analyzed by a variety of immuno-detection methods known in the art.

An efficacious means for in vitro assaying of cytotoxicity comprises the systematic exposure of a panel of tumor cells to selected plant extracts. Such assays and tumor cell lines suitable for implementing the assays are well known to those of skill in the art. Particularly beneficial human tumor cell lines for use in in vitro assays of anti-tumor activity include the human ovarian cancer cell lines SKOV-3, HEY, OCC1, and OVCAR-3; Jurkat T-leukemic cells; the MDA-468 human breast cancer line; LNCaP human prostate cancer cells, human melanoma tumor lines A375-M and Hs294t; and human renal cancer cells 769-P, 786-0, A498. A preferred type of normal cell line for use as a control constitutes human FS or Hs27 foreskin fibroblast cells.

In vitro determinations of the efficacy of a compound in killing tumor cells may be achieved, for example, by assays of the expression and induction of various genes involved in cell-cycle arrest (p21, p27; inhibitors of cyclin dependent kinases) and apoptosis (bcl-2, bcl-x_{L} and bax). To carry out this assay, cells are treated with the test compound, lysed, the proteins isolated, and then resolved on SDS-PAGE gels and the gel-bound proteins transferred to nitrocellulose membranes. The membranes are first probed with the primary antibodies (e.g., antibodies to p21, p27, bax, bcl-2 and bcl-x₁, etc.) and then detected with diluted horseradish peroxidase conjugated secondary antibodies, and the membrane exposed to ECL detection reagent followed by visualization on ECL-photographic film. Through analysis of the relative proportion of the proteins, estimates may be made regarding the percent of cells in a given stage, for example, the G0/G1 phase, S phase or G2/M phase.

Cytotoxicity of a compound to cancer cells also can be efficiently discerned in vitro using MTT or crystal violet staining. In this method, cells are plated, exposed to varying concentrations of the sample compounds, incubated, and stained with either MTT (3-(4,5-dimethylethiazol-2-yl)-2,5-diphenyle tetrazolium bromide; Sigma Chemical Co.) or crystal violet. MTT treated plates receive lysis buffer (20% sodium dodecyl sulfate in 50% DMF) and are subject to an additional incubation before taking an OD reading at 570 nm. Crystal violet plates are washed to extract dye with Sorenson's buffer (0.1 M sodium citrate (pH 4.2), 50% v/v ethanol), and read at 570-600 ran (Mujoo et al., 1996). The relative absorbance provides a measure of the resultant cytotoxicity.

Combinations of triterpenoid compounds and proteasome inhibitors can also be assayed in vitro for their effect on proteasome function. Those of skill in the art are familiar with methods for assaying proteasome function. For example, proteasome assays may be performed using a fluorometric assay that measures the hydrolysis of a labeled proteasome substrate such as SLLVY-AMC. The substrate is a five amino acid peptide attached to a fluor (4-amino-7-methylcoumarin) which, upon cleavage by the chymotrypsin-like activity of the proteasome, results in a fluorescent signal that can be measured and plotted over time. An example of another proteasome substrate known to those of skill in the art is BocLRR-AMC. The activity of the proteasome is reflected by the rate, or slope of the line. In this assay, the inhibition of proteasome activity by the combination of a triterpene and a proteasome inhibitor may be compared to that of either compound alone. Another method for assaying proteasome function is immunofluorescence using antibodies that recognize active proteasomes. For example, LMP2 antibodies specifically recognize the proteasome beta subunit. In addition, proteasome assay kits are commercially available from Biomol International LP.

(ii) In Vivo Assays

The present invention encompasses the use of various animal models. Here, the identity seen between human and mouse provides an excellent opportunity to examine the function of a potential therapeutic agent, for example, the compositions of the current invention. One can utilize cancer models in mice that will be highly predictive of cancers in humans and other mammals. These models may employ the orthotopic or systemic administration of tumor cells to mimic primary and/or metastatic cancers. Alternatively, one may induce cancers in animals by providing agents known to be responsible for certain events associated with malignant transformation and/or tumor progression.

Animal models for inflammatory disorders are also known to those of skill in the art. For example, mouse models for colitis include the DSS-induced colitis model, IL-10 knockout mouse, A20 knockout mouse, TNBS-induced colitis model, IL-2 knockout mouse, TCRalpha receptor knockout, and E-cadherin knockout.

Treatment of animals with test compounds will involve the administration of the compound, in an appropriate form, to the animal. Administration will be by any route the could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

It will be understood by those of skill in the art that therapeutic agents, including the compositions of the present invention, or combinations of such with additional agents, should generally be tested in an in vivo setting prior to use in a human subject. Such pre-clinical testing in animals is routine in the art. To conduct such confirmatory tests, all that is required is an art-accepted animal model of the disease in question. Any animal may be used in such a context, such as, e.g., a mouse, rat, guinea pig, hamster, rabbit, dog, chimpanzee, or such like. Studies using small animals such as mice are widely accepted as being predictive of clinical efficacy in humans, and such animal models are therefore preferred in the context of the present invention as they are readily available and relatively inexpensive, at least in comparison to other experimental animals.

The manner of conducting an experimental animal test will be straightforward to those of ordinary skill in the art. All that is required to conduct such a test is to establish equivalent treatment groups, and to administer the test compounds to one group while various control studies are conducted in parallel on the equivalent animals in the remaining group or groups. One monitors the animals during the course of the study and, ultimately, one sacrifices the animals to analyze the effects of the treatment.

Determining the effectiveness of a compound in vivo may involve a variety of different criteria. Such criteria include, but are not limited to, survival, reduction of tumor burden or mass, arrest or slowing of tumor progression, elimination of tumors, inhibition or prevention of metastasis, reduction of inflammation, increased activity level, improvement in immune effector function, and improved food intake.

The methods and composition of the present invention are useful in treating inflammation in a subject. One of ordinary skill in the art would be familiar with the wide range of techniques available of assaying for inflammation in a subject, whether that subject is an animal or a human subject. For example, inflammation can be measured by histological assessment and grading of the severity of inflammation. Other methods for assaying inflammation in a subject include, for example, measuring myeloperoxidase (MPO) activity, transport activity, and transcutaneous electrical resistance (TER). The effectiveness of a compound can also be assayed using tests that assess cell proliferation. For example, cell proliferation may be assayed by measuring 5-bromo-2′-deoxyuridine (BrdU) uptake. Yet another approach to determining the effectiveness of the compounds would be to assess the degree of apoptosis. Methods for studying apoptosis are well known in the art and include, for example, the TUNEL assay.

One of the most useful features of the present invention is its application to the treatment of cancer. Accordingly, anti-tumor studies can be conducted to determine the specific effects upon the tumor vasculature and the anti-tumor effects overall. As part of such studies, the specificity of the effects should also be monitored, including the general well being of the animals.

In the context of the treatment of solid tumors, it is contemplated that effective amounts of the compositions of the invention will be those that generally result in at least about 10% of the cells within a tumor exhibiting cell death or apoptosis. Preferably, at least about 20%, about 30%, about 40%, or about 50%, of the cells at a particular tumor site will be killed. Most preferably, 100% of the cells at a tumor site will be killed.

The extent of cell death in a tumor is assessed relative to the maintenance of healthy tissues in all of the areas of the body. It will be preferable to use doses of the compounds of the invention capable of inducing at least about 60%, about 70%, about 80%, about 85%, about 90%, about 95% up to and including 100% tumor necrosis, so long as the doses used do not result in significant side effects or other untoward reactions in the animal. All such determinations can be readily made and properly assessed by those of ordinary skill in the art. For example, attendants, scientists and physicians can utilize such data from experimental animals in the optimization of appropriate doses for human treatment. In subjects with advanced disease, a certain degree of side effects can be tolerated. However, patients in the early stages of disease can be treated with more moderate doses in order to obtain a significant therapeutic effect in the absence of side effects. The effects observed in such experimental animal studies should preferably be statistically significant over the control levels and should be reproducible from study to study.

Those of ordinary skill in the art will further understand that combinations and doses of the compounds of the invention that result in tumor-specific necrosis towards the lower end of the effective ranges may nonetheless still be useful in connection with the present invention. For example, in embodiments where a continued application of the active agents is contemplated, an initial dose that results in only about 10% necrosis will nonetheless be useful, particularly as it is often observed that this initial reduction “primes” the tumor to further destructive assault upon subsequent re-application of the therapy. In any event, even if upwards of about 40% or so tumor inhibition is not ultimately achieved, it will be understood that any induction of thrombosis and necrosis is nonetheless useful in that it represents an advance over the state of the patients prior to treatments. Still further, it is contemplated that a dose of the compounds of the invention which prevents or decreases the likelihood of either metastasis or de novo carcinogenesis would also be of therapeutic benefit to a patient receiving tire treatment.

As discussed above in connection with the in vitro test system, it will naturally be understood that combinations of agents intended for use together should be tested and optimized together. The compounds of the invention can be straightforwardly analyzed in combination with one or more chemotherapeutic drugs, immunotoxins, coaguligands or such like. Analysis of the combined effects of such agents would be determined and assessed according to the guidelines set forth above.

### J. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Effect of Avicins on the Expression of Heat Shock Proteins

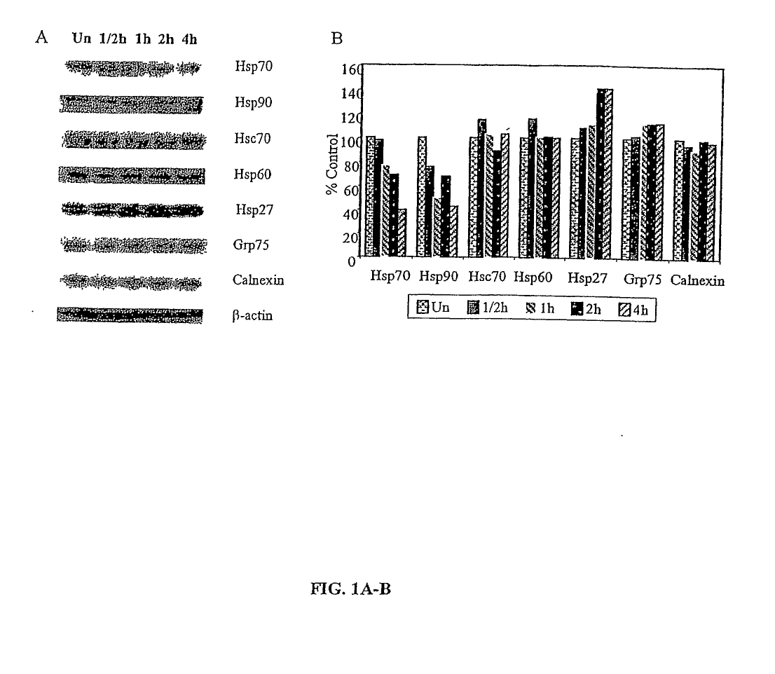

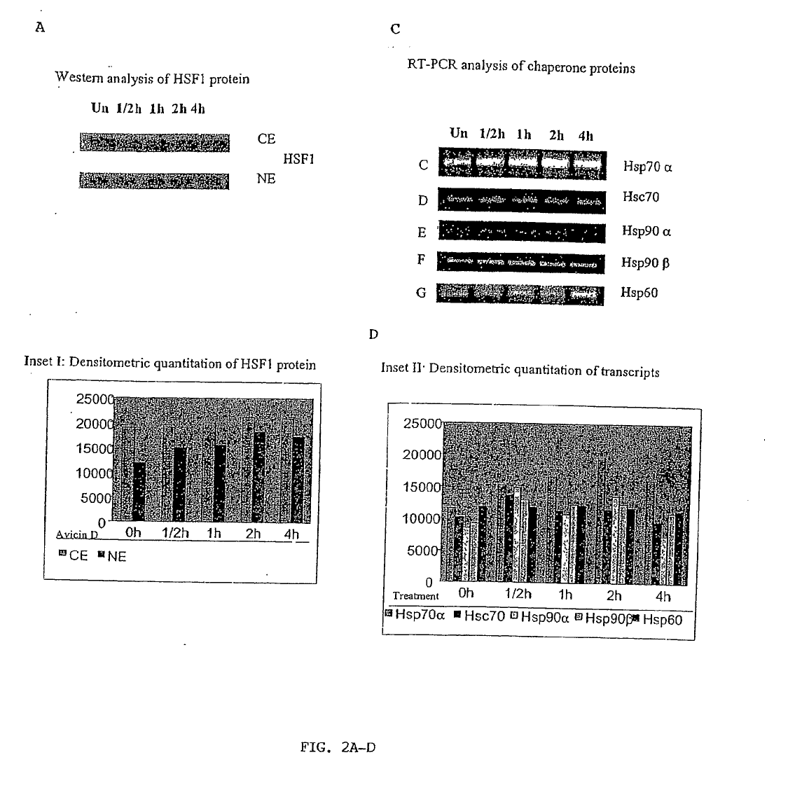

To study the effect of avicins on Hsps, the expression levels of various chaperone proteins in avicin D (1 μM) treated Jurkat cells were examined. As shown in FIGS. 1A and 1B, avicin D induced a significant decrease in the protein levels of Hsp70 and Hsp90 within one hour of treatment that persisted up to 4 hours. With the exception of Hsp27, which showed a modest increase (1.4 fold) at 2-4 hours of avicin D treatment, expression of other chaperone proteins like Hsc70, the mitochondrial localized Hsp60 and grp75, and the ER resident protein calnexin did not show any change, suggesting specificity of the action of avicins in the leukemia cells.

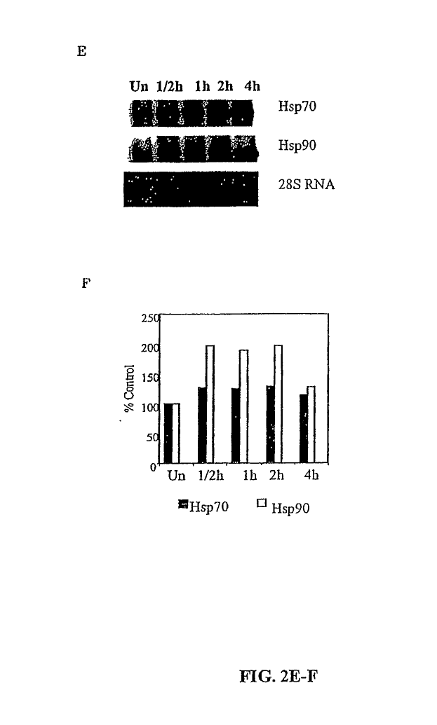

To understand the regulation of avicin-induced decrease in Hsps, Hsp transcription was also studied. Hsps are regulated at the transcriptional level via the heat shock factor (HSF1), which under unstressed conditions resides in the cytoplasm as an inactive monomer. Under stress, HSF1 undergoes oligomerization and nuclear translocation (Sarge et al., 1993), prior to the transcription of Hsp genes. Nuclear and cytoplasmic proteins were prepared from avicin treated cells to examine changes in HSF1 protein. No apparent change in the cytoplasmic content of HSF1 protein was detected, but avicin treatment (4 hours) induced a modest increase (˜1.5 fold) in the levels of nuclear HSF1 as determined by densitometric scanning (FIG. 2A and FIG. 2B).

RT-PCR was employed to see the effect of avicin D on the transcripts of heat shock proteins. A ˜1.6-fold increase in the Hsp70α and a ˜1.4-fold increase in the Hsp90β (FIG. 2C and FIG. 2D) transcripts were observed as early as 30 minutes after avicin treatment. The changes in the transcripts encoding Hsp90α, Hsc70, and Hsp60 were marginal (FIG. 2C and FIG. 2D). Northern blot analysis of Hsp70 (˜1.4 fold) and Hsp90 (˜2 fold) transcripts also revealed an increase in both of the transcripts (FIGS. 2E and 2F).

The increase in the levels of both nuclear HSF1 and Hsp transcripts (Hsp70 and Hsp90) are possibly due to removal of the feed-back inhibition of Hsp protein on HSF1. These results confirmed that the avicin-induced decrease in the Hsp70 protein is not at the level of transcription.

### Example 2

### Post-Transcriptional Regulation of Hsp70

The effect of lactacystin, an irreversible proteasomal inhibitor, on the avicin-induced decrease in Hsp70 and Hsp90 proteins was studied to determine if proteasomal degradation could be responsible for the decrease in Hsp70 and Hsp90 proteins.

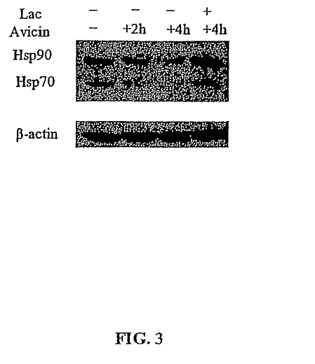

The cells that were treated with avicins for 2 and 4 hours showed a significant decrease in Hsp70 and Hsp90 proteins (FIG. 3) as compared with the untreated cells. However, pretreatment of Jurkat cells with lactacystin totally reversed the avicin-induced decrease in Hsp70 and Hsp90 proteins, showing proteasome-based degradation of Hsp70.

### Example 3

### Avicins Induce Ubiquitination

Since most proteins destined for proteasomal degradation are marked by their ubiquitination (Weissman, 2001), the involvement of the ubiquitin system in avicin-induced Hsp70 degradation was studied. An in vitro ubiquitination assay was performed using recombinant Hsp70 and histidine-tagged ubiquitin (his-ub) with cytoplasmic extracts of treated cells. As shown in FIG. 4A, the avicin-treated extracts induced a stronger ladder of his-ub-Hsp70 as compared to the extracts of the untreated cells, suggesting that avicins induce ubiquitination of Hsp70.

To establish an in vivo relevance, Jurkat cells transfected with a plasmid expressing a fusion protein of histidine-tagged-ubiquitin (his-ub) was treated with avicin D or lactacystin. The his-tagged proteins were affinity-purified and analyzed using anti-Hsp70 antibodies. FIGS. 4B and 4C shows a significant decrease (40%, p<0.05) in the levels of his-ub-Hsp70 protein band (−140 kDa) in avicin-treated cells for 2 and 4 hours, which was sensitive to lactacystin. The small amounts of his-ub-Hsp70 protein molecules synthesized in vivo, made it evident that the endogenous ubiquitin pool was competing with the his-ubiquitin for conjugation. Western analysis of the total CE using anti-Hsp70 antibodies showed similar change in Hsp70 protein as seen with the his-ub-Hsp70 fraction upon avicin treatment (FIG. 4B). Use of NEM during cellular extract preparation facilitated the visualization of additional bands of his-ub-Hsp70 (FIG. 5) around the prominent 140 kDa band. These results indicate that avicins induce ubiquitination and subsequent proteolytic degradation of Hsp70.

### Example 4

### Avicins Induce the E3α Ubiquitin Ligase

Ubiquitination involves three steps that utilize E1 (activating enzyme), E2 (conjugating enzyme), and E3 ligases (Weissman, 2001). Based on the importance of E3 ligases in carcinogenesis (Fang et al., 2003), the involvement of E3 ligase(s) in the degradation of Hsps was investigated. The Hsp70 amino acid sequence contains a putative caspase recognition motif starting at position 7 (“VGID”) followed by “L”, a destabilizing amino acid. Based on this observation, E3α ubiquitin ligase was selected for further investigation as it has been shown to have several confirmed and putative N-end rule substrates after the caspases cleave and expose the destabilizing amino acid (Varshavsky, 2003). In addition, Ditzel et al reported a connection between the ubiquitin system and apoptosis by demonstrating caspase mediated cleavage of DIAP1 followed by its ubiquitination by E3α ligase enzyme and its subsequent degradation.

Avicins induced a dramatic increase in the E3α protein with a peak at one hour of treatment (FIG. 6A). No significant change was observed in the levels of CHIP (carboxy terminus homology to Hsc/Hsp70 protein, FIG. 6A), another E3 ligase, under the same conditions thereby indicating the specificity of E3α induction by avicins.

To investigate if Hsp70 undergoes caspase-mediated cleavage followed by the degron pathway, zVAD was used to block the caspase activity. As shown in FIG. 6B, inhibition of caspases had no significant effect on the avicin-induced degradation of Hsp70, thereby ruling out the involvement of E3α in the caspase-mediated degradation of Hsp70. The ability of zVAD to block the caspase activity was monitored by examining the caspase 3 cleavage, and the protein-loading pattern was studied by probing the blot with anti-GAPDH antibodies (FIG. 6B).

Some reports suggest that the anti-apoptotic property of Hsp70 may be due to the presence of a conserved EEVD caspase recognition motif at the C-terminal end (Creagh et al., 2000). The inventors therefore looked at caspase 9 activation upon avicin treatment under these conditions. An increased cleavage of caspase 9 was observed at 2 hours of treatment (FIG. 6C). The activation of caspase 9 (at 2 hours) appears to closely follow the degradation of Hsp70, which occurs after 1 hour of avicin treatment in Jurkat cells (FIG. 1). The kinetics of the two events suggests that a decrease in Hsp70 is necessary for the activation of caspases.

### Example 5

### Role of E3α Ubiquitin Ligase in the Degradation of XIAP

A connection has been made between the ubiquitin system and apoptosis by demonstrating caspase mediated cleavage of DIAP1 followed by its degradation via the N-end rule pathway (Finley et al., 1984; Ditzel et al., 2003). The levels of inhibitor of apoptosis proteins (IAPs) known to have auto-ubiquitination activity, now have a second mechanism of regulation by the E3α degron pathway. Therefore, based on the discovery that avicins induce E3α (FIG. 6A), the effect of avicins on XIAP was studied.

Avicin-treated Jurkat cells showed a significant decrease in XIAP protein starting at 1 hour post treatment (FIG. 7A). Lactacystin blocked the avicin induced XIAP decrease, confirming a proteasome-based degradation of XIAP as shown in FIG. 7B. To explore if E3α regulates XIAP protein for which caspase activity is necessary, zVAD-fmk was used to block the caspases and monitor its effect on avicin D mediated XIAP degradation. Avicin-induced XIAP degradation was partially blocked (−22%) by zVAD-fmk (FIG. 7C, lane 4 and FIG. 7D), suggesting that besides the degron pathway (involving E3α ligase), other pathways (auto-ubiquitination) are involved in the degradation of XIAP. The observation that nearly 60% of XIAP is degraded by 1 hour (FIG. 7A) at the time of maximum induction of E3α elucidates its fractional involvement in degrading XIAP. However, the presence of several other proteins that could be targets of E3α ubiquitin ligase cannot be ruled out.

### Example 6

### Effect of Avicins on the Proteasomal Activity

The ubiquitin/proteasome machinery has been proposed to play a key role in the regulation of apoptosis. Specific inhibitors of proteasomes have been shown to induce apoptosis by accumulation of pro-apoptotic molecules and other less characterized mechanisms (Jesenberger and Jentsch, 2002). Therefore, the effect of avicin D on the proteasome function in Jurkat leukemia cells was investigated. A time dependent decrease in the 20S proteasomal activity was observed upon avicin D treatment with the maximum and significant decrease of 33% and 41% at 2 hours and 4 hours, respectively. (FIG. 8A). The decrease in the proteasomal activity from 2 hours matches with the protein conjugates observed in avicin D treated cell extracts, at around the same time (FIG. 8B). Recently, Sun et al. showed that caspase activation inhibits the proteasome function during apoptosis (Sun et al., 2004), a process that leads to accumulation of pro-apoptotic factors. The 30-40% decrease in proteasome activity during 2-4 hours of avicin treatment is in agreement with the observation of caspase 9 (FIG. 5C) and caspase 3 activation (FIG. 8C). It is, however, important to mention that the known anti-apoptotic proteins such as Hsp70 (FIG. 1), Hsp90 (FIG. 1), and XIAP (FIG. 7A) are degraded to a great extent, within 2 hours of avicin treatment when the proteasome activity shows only a marginal decrease.

### Example 7

### Avicins Cause Upregulation of Protein Ubiquitination in S. pombe Cells

Experiments were carried out to determine whether avicins affect protein ubiquitination in S. pombe cells. Wild type S. pombe cells were treated with 20 μg/ml avicin G and aliquots of the cell cultures were harvested between 30 minutes and 4 hours post-exposure to the drug. Cell extracts were then prepared and resolved by SDS-PAGE and subsequent immunoblotting to detect ubiquitinated proteins. As shown in FIG. 9, an increase in ubiquitinated proteins was apparent after 90 minutes of avicin G treatment and the levels of ubiquitinated proteins increased significantly with prolonged drug treatment.

An S. pombe mutant defective in function for the anaphase promoting complex (APC) was utilized to investigate whether the increase in levels of ubiquitinated proteins resulting from avicin G treatment was attributable to inhibition of 26S proteasome activity, upregulation of protein ubiquitination, or both. Two temperature sensitive 26S proteasome mutants, mts2-1 and mts3-1, exhibited sensitivities to avicin G that were-only slightly increased from wild-type S. pombe cells at their semi-permissive growth temperature of 26° C. (FIG. 10A). In contrast, an S. pombe mutant carrying a temperature sensitive mutation in the nuc2 gene (nuc2-663), which encodes an essential component of the APC mitotic ubiquitin ligase complex in S. pombe (Yamada et al., 1997), was markedly resistant to avicin G (FIGS. 10A and 10B). These results suggest that the increase in levels of ubiquitinated proteins that occurs in response to avicin G treatment may be attributable to the upregulation of protein ubiquitination, rather than to inhibition of 26S proteasome activity, an experimental conclusion similar to that achieved with human leukemia cells treated with avicin D.

### Example 8

### Effect of Avicin D on Other Leukemic/Lymphoma Cell-Lines and Fresh PBL from SS Patients

To rule out the possibility that the effects of avicins in Jurkat leukemia cells described above, could be cell-type specific, additional leukemic/lymphoma cells treated with avicin D were evaluated. Though the effects of avicin D on modulation of Hsp70 and XIAP vary at 4 hours in the different cell-lines tested (Jurkat, U937, MJ-1, and HH), a significant decrease in Hsp70 and XIAP appeared to be consistent at 24 hours avicin D post-treatment in all the cells (FIGS. 11A, 11B, and 11C). This observation suggests that the ability of avicins to regulate Hsp70 and XIAP is not restricted to a cell-type.

When primary peripheral blood lymphocytes (PBL) from Sezary syndrome (SS) patients were treated with avicin D for 24 hours, a decrease in both Hsp70 (25-35%) and XIAP (30-40%) proteins was observed (FIGS. 12A, 12B, and 12C). Interestingly, avicin D treatment also caused apoptosis in these CTCL cells. PBL from a normal blood sample treated with avicin D showed no significant change in the Hsp70 and XIAP proteins (FIG. 12D) and appeared to be resistant to apoptosis. Thus, avicins' ability to regulate the two anti-apoptotic proteins in various cells may contribute to its pro-apoptotic function.

### Example 9

### Experimental Procedures

### 1. Avicin D

Avicin D was isolated from the seedpods of A. victoriae as described in Haridas (2001).

### 2. Antibodies, Plasmids, Recombinant Proteins, and Cell Lines:

Human Jurkat T cell leukemia, monocytic U937 cells, and cutaneous T-cell lymphoma (CTCL) cell lines MJ (G11) and HH were obtained from American Type Culture Collection (Rockville, Md.) and grown in RPMI 1640 medium supplemented with 10% FBS and 2 mM glutamine.

Anti-Hsp70, anti-Hsp90, anti-Hsc70, anti-Hsp60, anti-HSF1, anti-β-actin, and anti-ubiquitin antibodies were purchased from StressGen. Anti-Ubr1, anti-calnexin, anti-grp75, and Protein A/G Agarose beads were purchased from Santa Cruz Biotechnology. Rabbit anti-CHIP antibodies were purchased from Oncogene Research Products. Anti-caspase 9, anti-caspase 3, and anti-XIAP antibodies were obtained from Cell Signaling. Anti-GAPDH mouse monoclonal antibodies were obtained from Ambion. Prestained protein markers were purchased from BioRad.

Primer sequences to perform RT-PCR were obtained from StressGen. The ProBond Nickel Agarose purification kit was purchased from Qiagen.

A plasmid expressing a fusion of GFP and histidine tagged ubiquitin (pDG268) for transient transfection of Jurkat T cells was a kind gift from Prof. Douglas Gray (Center for Cancer Therapeutics, Ottawa Regional Cancer Center). The his-Ub/GFP fusion is very efficiently processed in cells, and it is only the his-ub portion that gets conjugated to proteins (D. Gray, Personal communication).

Recombinant Hsp70 protein, ubiquitin, histidine tagged ubiquitin, and lactacystin were purchased from Sigma-Aldrich.

### 3. Treatment of the Cells:

Jurkat T cells (2 μg/ml=1 μM), U-937 (4 μg/ml), MJ (5 μg/ml), and HH cells (2.5 μg/ml) were treated for 0-24 hours with the indicated concentrations of avicin D. PBLs from the patients or normal blood were treated with 5 μg/ml of avicin D for 24 hours.

At the end of treatments, cells were harvested, washed with sterile ice-cold PBS and cytoplasmic extracts (CE) were prepared by lysing the cells in CE buffer containing 10 mM Hepes-Cl pH 7.5, 10 mM KCl, 0.1 mM EDTA, 0.1 mM EGTA, 0.3% NP40, and a protease inhibitor cocktail (Sigma). After centrifugation and separating the supernatant (CE proteins), the pellet was resuspended in a buffer containing 20 mM Hepes-Cl, 400 mM NaCl, 1 mM EDTA, 1 mM EGTA, and protease inhibitor cocktail (Sigma). The nuclear protein extraction proceeded for 30 min. on ice followed by centrifugation at 14,000 rpm for 5 min at 4° C. The clear supernatant containing nuclear proteins (NE) was collected, glycerol (10%) was added, and proteins stored at −80° C. until use.

### 4. Western Blot Analysis:

SDS-PAGE and immunoblot procedures were essentially performed as described (Sambrook, 1989). Briefly, cytoplasmic and nuclear proteins were resolved on SDS-PAGE, blotted on PVDF membranes (BioRad) and probed with various antibodies followed with anti-rabbit, anti-mouse antibody conjugated to horseradish peroxidase (HRP) from BioRad or HRP conjugated anti-goat antibody from Santa Cruz Biotechnology, corresponding to the primary antibody. Protein bands were detected using the ECL chemiluminescence kit from Amersham as per the manufacturer's protocol.

### 5. Northern Blot Analysis:

Total RNA from the control and avicin treated Jurkat T cells was made using Trizol (Invitrogen). Equal amounts of RNA were separated on form amide gels and transferred to nylon membranes (Hybond N+, Amersham) and UV cross-linked using UV Stratalinker (Stratagene). Staining the membranes with 0.03% methylene blue solution in 0.3% sodium acetate, pH 5.2, monitored equal loading. The DNA probes for Hsp70 and Hsp90 were purchased from StressGen as pUC plasmids and used according to the manufacturer's protocol. The DNA fragments were radiolabeled using a Nick Translation kit from Gibco BRL and [³²P] dCTP (Amersham). The membranes were exposed for autoradiography after hybridization using ExpressHyb (Clontech) solution at 58° C. for 1 hour and 5 washes, each of 20 minutes, with 5× SSC containing 0.1% SDS at 50° C.

### 6. RT-PCR:

Total RNA purified using Trizol method (Invitrogen) was subjected to DNAseI (RNAase free, Sigma Chemical Co.) treatment to remove any residual DNA, followed by heat inactivation and addition of 1 mM EDTA. Absence of genomic DNA was confirmed by performing PCR using Taq DNA polymerase. About 50-100 ng of purified total RNA was used in a one-step RT-PCR reaction kit from Invitrogen in a Techne Genius machine. The samples were separated on 0.8% agarose-TBE gels and viewed by staining with ethidium bromide.

### 7. Densitometric Analysis:

Quantitation of proteins (western) and transcripts (RT-PCR) was performed using the NIH 1.61 image software.

### 8. In Vitro Ubiquitination:

Ubiquitination assays were performed as described (Firestein and Feuerstein, 1998) with few modifications using recombinant bovine Hsp70 and N-terminal histidine-tagged ubiquitin (his-ub). About 0.5 μg of Hsp70 and 4 μg of his-ub were incubated in a buffer containing 50 mM Tris-Cl pH 7.5, 2.5 mM MgCl₂, 0.05% NP 40, 0.5 mM DTT, 5 mM ATP, 4 μM MG132, and ATP regenerating system containing 10 mM creatine phosphate, 0.1 μg/ml of creatine kinase, and about 50 μg of CE proteins. The reaction was carried out for 1 hour at 30° C. and the products were subjected to nickel agarose chromatographic purification to purify histidine-tagged proteins as per manufacturer's protocol (Qiagen). The affinity-purified proteins were prepared for SDS-PAGE and western analysis using anti-Hsp70 antibodies.

### 9. Transient Transfection:

Jurkat T-cells were transfected with a plasmid pDG268 that expresses a fusion protein of histidine-tagged human ubiquitin and enhanced GFP. Transfection was performed using μm ax a Biosystems kit and their protocol. After 24 hours of transfection, cells were harvested, resuspended at a density of 10⁶ cells/ml before treatment with lactacystin or avicin D.

### 10. In Vivo Ubiquitination Activity:

Jurkat T cells transfected with the his-ub plasmid construct were treated with lactacystin (10 μM) or with avicin D (1 μM) for 4 hours. Cells were harvested and CE prepared as described above. The his-ub containing proteins (250 μg) were purified using nickel agarose beads as suggested by the manufacturer (Qiagen). The affinity purified histidine-tagged proteins were separated on SDS-PAGE and analyzed on western blots for ub-Hsp70 proteins.

### 11. 20S Proteasomal Assay:

Jurkat T cells were treated with 1 μM of avicin D for 0-4 hours. Proteasomal extracts (PE) were prepared as described previously (18) in a buffer containing 50 mM Hepes pH 8, 5 mM EGTA, 0.3% NP40, and 10% glycerol. The assay reaction contained 20 mM Tris-Cl pH7.2, 0.1 mM EDTA, 1 mM β-mercaptoethanol, 5 mM ATP, 20% glycerol, 0.02% SDS, and 0.04% NP40. About 10 μg of the PE proteins and BocLRR-AMC (0.1 mM), which allows measurement of the trypsin-like activity of proteasomes, was used as substrate. The reaction was carried out at 30° C. for 30 minutes and the fluorescence was read at 380 nm (excitation) and 460 nm (emission) in a Perkin Elmer HTS 7000 Plus, Bioassay Reader.

### 12. Statistical Analysis:

Statistical significance of differences observed in the proteasomal activity in avicin treated cells compared with the untreated cells was determined by using an unpaired Student t test. The minimum level of significance was a P<0.05.

### 13. Yeast Strains and Manipulations:

Schizosaccharomyces pombe strains used were wild-type strains SP870 (h⁹⁰ ade6-210 leu1-32 ura4-D18), SP870D (h⁹⁰ ade6-210 leu1-32 ura-4-D18/h⁹⁰), and CHP428 (h⁺ ade6-M210 his 7-366 leu1-32 ura-4-D18). S. pombe mutant lines used were mts2-1 (h⁻ leu1-32 ura-4-D18 mts2-1), mts3-1 (h⁻ leu1-32 mts3-1), and nuc2-663 (h⁻ leu1-32 nuc2-663).

Standard yeast culture media and genetic methods were used (Alfa et al., 1993; Rose et al., 1990). S. pombe cultures were grown in either YEAU (0.5% yeast extract, 3% dextrose, 75 mg/ml adenine, 75 mg/ml uracil) or synthetic minimal medium (EMM) with appropriate supplements.

14. Detection of Ubiquitinated Proteins in S. pombe

S. pombe cultures were lysed with glass beads in PEM buffer (100 mM PIPES, 1 mM EGTA, 1 mM MgSO₄, pH 6.9) containing 4 mM benzamide, 10 μM E64, 50 μM leupeptin, 1 μM pepstatin, 1 mM phenylmethanesulfonyl fluoride, and 2 μg/ml aprotinin essentially as described in (Yen et al., 2003). Equal amounts of protein were resolved by SDS-PAGE and subsequent immunoblotting using anti-ubiquitin mouse monoclonal antibody (Stressgen Biotechnologies).

All of the composition and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the claims.

### REFERENCES

The following references, to the extent that they provide exemplary procedural or other details supplementary to those set forth herein, are specifically incorporated herein by reference.
U.S. Pat. No. 3,070,623
U.S. Pat. No. 3,070,624
U.S. Pat. No. 5,049,388
U.S. Pat. No. 5,607,915
U.S. Pat. No. 6,444,233
U.S. patent application Ser. No. 09/992,556
Alfa et al., In: Experiments with Fission Yeast: A Laboratory Course Manual, Cold Spring Harbor Laboratory Press, Cold Spring Harbor, N.Y., 1993.
Beere and Green, Trends Cell Biol, 11:6-10, 2001.
Beere et al., Nat. Cell Biol, 2:469-475, 2000.
Belgian Patent 753773
Benhar et al., EMBO Rep., 3:420-425, 2002.
British Patent 1346871
Chauhan et al., Blood, 103:3158-3166, 2004.
Creagh et al., Exp. Cell Res., 257:58-66, 2000.
Creagh et al., Leukemia, 14:1161-1173, 2000.
Ditzel et al., Nat. Cell Biol, 5:467-473, 2003.
Doll, et al., Lancet., 1:793, 1962.
Fang et al., Semin. Cancer Biol, 13:5-14, 2003.
Feder and Hofmann, Anna. Rev. Physiol, 61:243-282, 1999.
Firestein and Feuerstein, J. Biol. Chem., 273:5892-5902, 1998.
Hanausek et al., Proc. Natl. Acad. Sci. USA, 98:11551-11556, 2001.
Haridas et al., J. Clin. Invest., 113:65-73, 2004.
Haridas et al., Proc. Natl. Acad. Sci. USA, 98:11557-11562, 2001.
Haridas et al., Proc. Natl. Acad. Sci. USA, 98:11557-11562, 2001.
Haridas et al., Proc. Natl. Acad. Sci. USA, 98:5821-5826, 2001.
Hershko et al., Annu. Rev. Biochem., 67:425-479, 1998.
Hideshima et al., cancer Res., 61:3071-3076, 2001.
Holcik et al., Apoptosis, 6:253-261, 2001.
Hostettmann et al., In: Saponins, Cambridge University Press, 1-548; 1995.
Huang et al., Zhongueo Yaoii Xuebao, Chem. Abstract No. 98100885, 3:286-288, 1982.
Inoue et al., Chem. Pharm. Bull, 6(2):897-901,1986.
Japanese Pub. JP-A-44-32798
Jayatilake et al., J. Nat. Prod., 66:779-83, 2003.
Jesenberger and Jentsch, Nat. Rev. Mol. Cell. Biol, 3:112-121, 2002.
Jesenberger and Jentsch, Nat. Rev. Mol Cell Biol, 3:112-121, 2002.
Jolly and morimoto, J. Natl. Cancer Inst., 92:1564-1572, 2000.
Kasiwada et al., J. Org. Chem., 57:6946-6953, 1992.
Kisselev and Goldberg, Chem. Biol, 8:739-758, 2001.
Koepp et al., Cell, 97:431-434, 1999.
Kong et al., Phytochemistry, 33:427-430, 1993.
Lee and Goldberg, Mol. Cell. Biol, 18:30-38, 1998.
Mahato & Sen, Phytochemistry, 44:1185-1236, 1997.
McClintock, Science, 226:792-801, 1984.
Meng et al., Proc. Natl. Acad. Sci. USA, 96:10403-10408, 1999.
Mitsiades et al., Proc. Natl. Acad. Sci. USA, 99:14374-14379, 2002.
Mujoo et al., Cancer Res., 61:5486-5490, 2001.
Mujoo et al., Cancer Res., 61:5486-5490, 2001.
Mujoo et al., Oncogene, 12:1617-1623, 1996.
Myung et al., Med. Res. Rev., 21(4):245-273, 2001.
Nagamoto et al., Planta Medica., 54:305-307, 1988.
Nylandsted et al., Proc. Natl. Acad. Sci. USA, 97:7871-7876, 2000.
Pagano et al., Science, 269:682-685, 1995.
Palombella et al., Proc. Natl. Acad. Sci. USA, 95:15671-15676, 1998.
Pisha et al., Nature Medicine, 1:1046-1051, 1995.
Ravagnan et al., Natl. Cell Biol, 3:839-843, 2001.
Remington's Pharmaceutical Sciences 15th Edition, 33:624-652, 1990
Remington's Pharmaceutical Sciences, 16th Ed. Mack Publishing Company, 1980
Rose et al., In: Methods in Yeast Genetics: A Laboratory Course Manual., Cold Spring Harbor Laboratory, Cold Spring Harbor, N.Y., 1990.
Saleh, at al., Nat. Cell Biol., 2:476-483, 2000.
Sambrook et al., In: Molecular Cloning: A Laboratory Manual., Cold Spring Harbor Laboratory Press, NY, Vol 1,2,3,1989.
Sarge et al., Mol. Cell. Biol, 13:1392-1407, 1993.
Schimmer et al., Cancer Cell, 5:25-35, 2004.
Sun et al., Mol. Cell, 14:81-93, 2004.
Suzuki et al., J. Biol. Chem., 276:27058-27063, 2001.
Tamm et al., Clin. Cancer Res., 6:1796-1803, 2000.
Thompson et al., Cancer Epidemiol. Biomarker Prevent., 1:597-602, 1992.
Tomas-Barbaren et al., Planta Medica., 54:266-267, 1988.
Varshavsky, Nat. Cell Biol, 5:373-376, 2003.
Weissman, Nat. Rev. Mol. Cell. Biol, 2:169-178, 2001.
Wendt et al. Agnew. Chem. Int. Ed. Engl., 39:2812-2833, 2000.
Wyllie, Anticancer Res., 5:131-136, 1985.
Yamada et al., J. Cell Set, 110:1793-1804, 1997.
Yang and Yu, FASEB J., 17:790-799, 2003.
Yen et al., J. Biol. Chem., 278:30669-30676, 2003.

1. A method of inducing apoptosis in a malignant cell comprising contacting the malignant cell with a natural triterpenoid and a proteasome inhibitor.

2. The method of claim 1, wherein the natural triterpenoid is a plant-derived triterpenoid.

3. The method of claim 2, wherein the plant-derived triterpenoid is derivable from a plant of the Acacia genus.

4. The method of claim 3, wherein the plant-derived triterpenoid is derivable from Acacia victoriae.

5. The method of claim 1, wherein the natural triterpenoid is an avicin.

6. The method of claim 1, wherein the proteasome inhibitor is a peptide aldehyde, a peptide boronate, a peptide vinyl sulfone, a peptide epoxyketone, a lactacystin, or a lactacystin derivative.

7. The method of claim 1, wherein the malignant cell is a cancer cell.

8. The method of claim 7, wherein the cancer cell is an ovarian cancer cell, a pancreatic cancer cell, a renal cancer cell, a prostate cancer cell, a melanoma cell, or a leukemia cell.

9. A method treating a subject with a malignancy comprising administering to said subject a natural triterpenoid and a proteasome inhibitor.

10. The method of claim 9, wherein the subject is a mammal.

11. The method of claim 10, wherein the mammal is a human.

12. The method of claim 9, wherein said administering is via a route selected from the group consisting of intratumoral injection, intravenous injection, oral, and topical.

13. The method of claim 9, wherein the natural triterpenoid is a plant-derived triterpenoid.

14. The method of claim 13, wherein the plant-derived triterpenoid is derivable from a plant of the Acacia genus.

15. The method of claim 14, wherein the plant-derived triterpenoid is derivable from Acacia victoriae.

16. The method of claim 9, wherein the natural triterpenoid is an avicin.

17. The method of claim 9, wherein the proteasome inhibitor is a peptide aldehyde, a peptide boronate, a peptide vinyl sulfone, a peptide epoxyketone, a lactacystin, or a lactacystin derivative.

18. A method treating a subject with an inflammatory disorder comprising administering to said subject a natural triterpenoid and a proteasome inhibitor.

19. The method of claim 18, wherein the subject is a mammal.

20. The method of claim 19, wherein the mammal is a human.

21. The method of claim 18, wherein said administering is via a route selected from the group consisting of intratumoral injection, intravenous injection, oral, and topical.

22. The method of claim 18, wherein the natural triterpenoid is a plant-derived triterpenoid.

23. The method of claim 22, wherein the plant-derived triterpenoid is derivable from a plant of the Acacia genus.

24. The method of claim 23, wherein the plant-derived triterpenoid is derivable from Acacia victoriae.

25. The method of claim 18, wherein the natural triterpenoid is an avicin.

26. The method of claim 18, wherein the proteasome inhibitor is a peptide aldehyde, a peptide boronate, a peptide vinyl sulfone, a peptide epoxyketone, a lactacystin, or a lactacystin derivative.

27. The method of claim 18, wherein the inflammatory disorder is an autoimmune disorder.

28. A pharmaceutical composition comprising a natural triterpenoid and a proteasome inhibitor in a pharmacologically acceptable buffer, solvent or diluent.

29. The pharmaceutical composition of claim 28, wherein the natural triterpenoid is further defined as Avicin D and the proteasome inhibitor is further defined as PS-341 (bortezomib).

30. The pharmaceutical composition of claim 28, wherein the natural triterpenoid is further defined as Avicin G and the proteasome inhibitor is further defined as PS-341 (bortezomib).

31. The pharmaceutical composition of claim 28, wherein the natural triterpenoid is further defined as Avicin B and the proteasome inhibitor is further defined as PS-341 (bortezomib).

32. A method of treating cell proliferative disease in a subject comprising, administering an effective amount of a natural triterpenoid compound and an effective amount of a proteasome inhibitor.

33. The method of claim 32, wherein the natural triterpenoid compound and the proteasome inhibitor are administered simultaneously.

34. The method of claim 32, wherein the natural triterpenoid compound and the proteasome inhibitor are administered sequentially.

35. The method of claim 32, wherein the cell proliferative disease is a cancer.

36. The method of claim 35, wherein in the cancer is an ovarian cancer, a pancreatic cancer, a renal cancer, a prostate cancer, a melanoma, or a leukemia.

37. The method of claim 35, wherein the cancer is multiple myeloma.

38. The method of claim 32, wherein the natural triterpenoid is a plant-derived triterpenoid.

39. The method of claim 38, wherein the plant-derived triterpenoid is derivable from a plant of the Acacia genus.

40. The method of claim 39, wherein the plant-derived triterpenoid is derivable from Acacia victoriae.

41. The method of claim 32, wherein the natural triterpenoid is an avicin.

42. The method of claims 41, wherein the avicin is Avicin B, Avacin G or Avacin D.

43. The method of claim 32, wherein the proteasome inhibitor is a peptide aldehyde, a peptide boronate, a peptide vinyl sulfone, a peptide epoxyketone, a lactacystin, or a lactacystin derivative.

44. The method of claim, 43 wherein the proteasome inhibitor is PS341 (bortezomib).

45. The method of claim 37, wherein the proteasome inhibitor is PS341 (bortezomib) and the natural triterpenoid is an avicin.

46. The method of claims 45, wherein the avicin is Avicin B, Avicin G or Avicin D.

47. The method of claim 7 wherein the cancer cell is a multiple myeloma cell.
